(19) United States
(12) Patent Application Publication
(10) Pub. No.: US 2024/0277450 A1
(43) Pub. Date: Aug. 22, 2024

(54) METHODS AND SYSTEMS FOR INTERPROXIMAL ADJUSTMENT

(71) Applicant: Align Technology, Inc., San Jose, CA (US)

(72) Inventors: Denis SHERSTENNIKOV, Moscow (RU); Andrey CHEKHONIN, Moscow (RU); Semen LAZAREV, Moscow (RU)

(21) Appl. No.: 18/582,576

(22) Filed: Feb. 20, 2024

Related U.S. Application Data
(60) Provisional application No. 63/485,879, filed on Feb. 17, 2023.

(51) Int. Cl. A61C 7/00; G06T 7/00; G06T 7/10; G16H 20/00

(52) U.S. Cl. CPC A61C 7/002; G06T 7/0012; G06T 7/10; G16H 20/00; G06T 2207/30036

(57) ABSTRACT

Methods, apparatuses, and systems for generating dental aligners and treatment plans for dental aligners that determine the benefit of interproximal adjustment procedures, such as interproximal reduction (IPR) and interproximal spacing procedures. These methods and systems may automatically evaluate a patient's dentition (e.g., upper and lower jaw) to generate recommendations for performing an interproximal adjustment procedure.

### CLAIM OF PRIORITY

This patent application claims priority to U.S. Provisional Patent Application No. 63/485,879, titled “METHODS AND SYSTEMS FOR INTERPROXIMAL ADJUSTMENT,” and filed on Feb. 17, 2023, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate generally to orthodontic treatment planning and associated dental appliances used in treatment, and more particularly to predicting whether one or more interproximal adjustment procedures would be clinically beneficial as part of a treatment plan.

### BACKGROUND

Treatment planning may be used in orthodontic treatments to accomplish a desired dental outcome. In general, the goal of an orthodontic treatment plan is to improve the appearance and function of a patient's teeth. In some cases, orthodontic treatments may involve the use of a series of patient-removable appliances (e.g., orthodontic aligners, palatal expanders, etc.), to treat dental malocclusions by incrementally repositioning the teeth. Treatment planning is typically performed in conjunction with the dental professional (e.g., dentist, orthodontist, dental technician, etc.) using treatment planning software to generate a model of the patient's teeth in a final target configuration and partition the treatment plan into a number of intermediate stages (steps) corresponding to individual appliances that are worn sequentially. This process may be interactive, adjusting the staging, and in some cases the final target position, based on constraints on the movement of the teeth and the dental professional's preferences.

In some cases, additional dental procedures in addition to those implemented by removable appliances may be beneficial to achieve a desired result. For instance, a patient's dentition may have size discrepancies between upper and lower teeth that are addressable using interproximal reduction (IPR), which involves the mechanically removing enamel from between the teeth, for example, to prevent overcrowding of adjacent teeth. Another example procedure involves providing additional space between adjacent teeth that are relatively small so that they provide a proper bite with teeth of the opposing jaw, and then adding a filler material (e.g., composite, crown or veneer material) between the teeth for aesthetics. It can be difficult to determine whether such additional procedures are necessary or beneficial to a particular patient's condition and treatment goals due to the many factors that go into determining treatment plans. For example, conventional treatment planning software may result in treatment plans that include IPR and/or increasing the interproximal spaces between teeth when such procedures do not necessarily provide the best overall benefits to the patient.

Successful treatment planning may benefit from an accurate prediction as to whether an interproximal adjustment procedure (e.g., IPR and/or increasing interproximal spaces) to compensate for tooth size and/or bite discrepancies is necessary or preferable to achieve a desired outcome.

### SUMMARY OF THE DISCLOSURE

Described herein are apparatuses, systems, and methods for planning and/or performing an orthodontic treatment plan including determining whether one or more of interproximal adjustment procedures should be performed as part of an orthodontic treatment. The orthodontic treatment may be performed by one or a series of removable dental appliances (e.g., aligners). Thus, also described herein are dental appliances (which may include a series of dental appliances), methods of making the dental appliances including any of these methods, and methods of using the dental appliances.

In general, the methods and apparatuses (e.g., systems) described herein may include determining if an orthodontic treatment will benefit from one or more of interproximal adjustment procedures, such as an interproximal reduction (IPR) and/or an interproximal spacing procedure.

A dental professional may use the prediction results to determine whether to integrate the additional dental procedure(s) into the orthodontic treatment plan. Alternatively, the prediction may automatically be integrated into the treatment plan. If integrated into the treatment plan, the treatment plan may include repositioning one or more teeth to account for the planned additional dental procedure(s).

The additional dental procedures may include interproximal adjustment procedures involving areas between adjacent teeth. Examples of interproximal adjustment procedures may include interproximal reduction (IPR) and interproximal spacing procedures. IPR procedures are typically used to compensate for overcrowded teeth and involve removing enamel from between the teeth. Interproximal spacing procedures are used to increase space between adjacent teeth to provide a proper bite, and is typically followed up with an additive process where tooth-like material is bonded to one or both of the adjacent teeth to compensate for the relatively small size of the teeth/tooth.

Methods may include dividing the teeth of the upper and lower jaws into posterior and anterior regions (also referred to as sections or segments). Each of the posterior segments may include molars and premolars, and each of the anterior segments may include incisors and canine teeth. For example, posterior segments may include an upper left posterior segment (left side of the upper jaw), an upper right posterior segment (right side of the upper jaw), a lower left posterior segment (left side of the lower jaw), and a lower right posterior segment (right side of the lower jaw). The anterior segments may include an upper anterior segment (of the upper jaw) and a lower anterior segment (of the lower jaw).

The patient's teeth are evaluated to determine one or more measurable clinical factors that may affect the treatment plan. Clinical factors may include a degree of overjet (if any), a molar bite class, a canine bite class, a degree of Bolton discrepancy (if any), the presence and number of missing teeth (if any), and the presence and number of primary teeth (if any). These clinical factors, along with expected (or desired) aspects of the target dentition, may be used to predict whether interproximal adjustment procedures would be advantageous in each of the posterior and anterior segments as part of the treatment plan. A user (e.g., dental professional) may then choose to implement an interproximal adjustment procedure in generating an orthodontic treatment plan based on the prediction.

In previous approaches of dental planning, the dental professional chooses to either allow or not allow the use of IPR in a treatment plan. If the dental professional chooses to allow the use of IPR, the treatment planning software typically calculated and optimized a treatment path based on many factors together with the option to use IPR. Often, this resulted in treatment plans that include IPR to make only minor improvements or to improve lesser treatment goals. The methods and apparatuses described herein advantageously provide a way for a user (e.g., dental professional) to predict whether any interproximal adjustment procedure would be beneficial separately and before full calculation of a treatment plan, thereby providing a more accurate prediction. In practice, this may reduce the incorporation of unnecessary interproximal adjustment procedures in treatment plans without sacrificing clinical goals of the treatment plans. The methods discussed herein may also allow the user to easily review all the factors used in making the predictions, and to modify input that influences how the prediction is made, thereby providing better control over the treatment planning process.

In particular, the methods and apparatuses described herein represent a marked improvement over other methods and systems by increasing the speed, accuracy and transparency, and therefore improving the operation of such systems. This is due, at least in part, by grouping (or dividing) the jaws into particular segments (also referred to herein as ‘regions’) of teeth; both the upper and lower jaw may be grouped into segment including groups of teeth and these segments may be separately processed. Described herein are method and apparatuses that group the teeth from the digital model into particular segments that allow the system (e.g., computer-based system) to process the otherwise complex analysis within a fixed tie period (including within a real- or near-real time period).

These methods and apparatuses may analyze a digital model of a patient's dentition, including the upper and lower jaws, and may incorporate both clinical characteristics of a particular patient's case (e.g., the patient's initial bite class, the effects of any missing or extracted teeth, the effect of tooth width, including the presence or absence of any Bolton excess, and/or the presence or absence of any positive or negative overjet), as well as any prescribed settings general to a particular user (e.g., clinician, dentist, orthodontist, etc.) treating the patient and/or prescribed settings specific to the patient. These prescribed settings may include one or more of: the user's allowance for IPRs between particular teeth or segments of teeth (e.g., within or between one or more segments), the user's allowance for spaces between particular teeth or segments of teeth (e.g., within or between one or more segments), the user's goals for bite class (e.g., improving bite call, maintaining bite class, etc.), and/or the user's goals for overjet (e.g., maintaining overjet or not).

Because the decision of whether to use an interproximal adjustment procedure is made before the calculation of a treatment plan, the resulting treatment plan may more accurately reflect the treatment goals. In addition, because a segment-based prediction model is used, where separate segments of the dentition (anterior and posterior segments) are analyzed for IPR and/or spaces, this results in less overall computing processing time compared to larger optimization programs based on the overall patent's dentition.

In some examples, the prediction model is based on a decision tree model where decisions and possible consequences are determined based on a combination of various clinical factors and aspects of the expected target dentition. Each of the segments of the dentition (posterior and anterior segments) may be analyzed independently by a decision tree. The posterior segments may include a different sequence of decisions than the sequence of decisions for anterior segments. Advantages of a decision tree model include the speed in which the prediction may be made, small burden on computer resources (e.g., processing and memory) compared to some other computational models, and accessibility by a user (e.g., presentable and easily modifiable). For example, the prediction logic may be presented explicitly via flowcharts, therefore it can be understood, visualized and changed in a straightforward way. Further, the prediction may be made within a discrete time period, for example, compared to some iterative processes or trial and error processes which may grow indefinitely.

The prediction models described herein have been shown to provide about a 10% improvement of automatic treatments per post-factum check of production data. In addition, manual assignment of IPRs and interproximal spaces may be avoided, making the work of CAD designers easier.

In some examples, an orthodontic treatment planning system includes: an intraoral scanning device configured to direct radiation from one or more radiation sources toward teeth of an upper jaw and a lower jaw within an oral cavity, the intraoral scanning device including one or more detectors configured to collect images of the teeth; and a computing device including a non-transitory computing device readable medium having instructions stored thereon that are executable by a processor to cause the computing device to: generate a digital model including teeth of the upper and lower jaw based on the collected images; predict whether one or more interproximal adjustment procedures would be beneficial as part of an orthodontic treatment plan, wherein the predicting includes: dividing the teeth of the upper and lower jaws into posterior segments and anterior segments, wherein each of the posterior segments includes at least one molar and at least one premolar, wherein each of the anterior segments includes at least one incisor and at least one canine tooth; and estimating whether one or both of an interproximal reduction (IPR) and an interproximal spacing procedure in each of the posterior and anterior segments would be beneficial as part of the treatment plan, wherein the estimating is based on one or more of: a presence of missing teeth, a presence of primary teeth, a degree of overjet, a bite classification of the teeth, and a degree of Bolton excess of the teeth; and output a recommendation as to whether an IPR or an interproximal spacing procedure on one or both of the posterior and anterior segments would be beneficial based on the prediction.

The posterior segments may include an upper left posterior segment, a lower left posterior segment, an upper right posterior segment, and a lower right posterior segment, and wherein the anterior segments include an upper anterior segment and a lower anterior segment. The estimating may be based on one or more of: width discrepancies between the upper and lower left posterior segments, between the upper and lower right posterior segments, and between the upper and lower jaws; and an initial bite class of opposing canine teeth in the digital model. The initial bite class or predicted bite class of opposing canine teeth may be class I, class II or class III. The estimating may be based on one or more of: the presence of missing or extracted canine teeth in the digital model; and a predicted bite class of opposing canine teeth after implementation of the orthodontic treatment plan. The predicted bite class of opposing canine teeth may be determined for opposing left canine teeth and opposing right canine teeth. The predicted bite class of opposing canine teeth may be determined based on whether one or both of the opposing canine teeth are missing or expected to be extracted during the orthodontic treatment. The predicted bite class of opposing canine teeth may be determined based on whether one or more incisors is expected to be extracted during the orthodontic treatment. The predicted bite class of opposing canine teeth may be determined based on whether the upper and lower anterior segments include a mixed dentition, where the mixed dentition includes a combination of primary and permanent teeth. The estimating may be based on a degree of Bolton excess of opposing anterior teeth. The estimating may be based whether the degree of Bolton excess of opposing anterior teeth is greater than a predetermined value. The estimating may be based on whether an overjet is to be maintained after the orthodontic treatment. The estimating may be based on whether an initial anterior tooth distribution includes a negative overjet. The estimating may be based whether a width discrepancy of upper and lower left posterior segments is outside of a first predetermined tolerance, whether a width discrepancy of upper and lower right posterior segments is outside of the first predetermined tolerance, and whether a width discrepancy of the upper and lower jaws is outside a second predetermined tolerance. The instructions may further cause the computing device to receive instructions, from a user via a user interface, to perform the predicting. The instructions may further cause the computing device to use the recommendation to generate the orthodontic treatment plan, wherein the orthodontic treatment plan includes a series of treatment stages each corresponding to an orthodontic appliance. Each of the orthodontic appliances may be a dental aligner shaped to resiliently apply repositioning forces on the teeth according to a treatment stage of the orthodontic treatment plan. The orthodontic treatment plan may be based on moving the teeth toward target teeth positions. The recommendation may be used to generate multiple orthodontic treatment plans.

In some examples, a method includes: receiving a digital model including teeth of an upper jaw and a lower jaw; predicting whether one or more interproximal adjustment procedures would be beneficial as part of an orthodontic treatment plan, wherein the predicting includes: dividing the teeth of the upper and lower jaws into posterior segments and anterior segments, wherein each of the posterior segments includes at least one molar and at least one premolar, wherein each of the anterior segments includes at least one incisor and at least one canine tooth; and estimating whether one or both of an interproximal reduction (IPR) and an interproximal spacing procedure in each of the posterior and anterior segments would be beneficial as part of the treatment plan, wherein the estimating is based on one or more of: a presence of missing teeth, a presence of primary teeth, a degree of overjet, a bite classification of the teeth, and a degree of Bolton excess of the teeth; and outputting a recommendation as to whether an IPR or an interproximal spacing procedure on one or both of the posterior and anterior segments would be beneficial based on the predicting.

For example, described herein are methods comprising: receiving a digital model including teeth of an upper jaw and a lower jaw; grouping teeth of the digital model into a plurality of segment, including at least two posterior segments and at least one anterior segment for each of the upper jaw and the lower jaw, wherein each of the posterior segments includes at least one molar and at least one premolar, and each of the anterior segments includes at least one incisor and at least one canine tooth; generating a segment recommendation for each segment of the plurality of segments indicating if one or more of interproximal adjustment procedures should be performed as part of an orthodontic treatment plan, based on one or more of: a presence of missing teeth, a presence of primary teeth, a degree of overjet, a bite classification of the teeth, and a degree of Bolton excess of the teeth; and outputting the segment recommendations.

The one or more of interproximal adjustment procedures may include an interproximal reduction (IPR) and/or an interproximal spacing procedure.

Outputting the segment recommendations comprises displaying the segment recommendations in a graphical user interface for approval by a user. In some examples outputting may include storing and/or transmitting the segment recommendations either locally or remotely (including on the cloud). Segment recommendations may be combined. The segment recommendations may be encoded, e.g., as an array or description, including text and/or numeric, and/or images. In some examples, the segment recommendations may be an array indexed by the patient's teeth or the regions between the patient's teeth. In some examples the segment recommendations may be graphical, and may include an annotation of the digital model (which may be annotated by include text, graphical, and/or numeric data or metadata).

In some examples, the method includes modifying the orthodontic treatment plan based on the segment recommendations. In some examples generating the segment recommendation comprises applying prescribed settings associated with a patient, wherein the digital model is specific to the patient. For example, the prescribed setting may include one or more of: allowance for IPRs on segment, allowance for spaces on segment, bite class goal, and overjet goal.

In some examples the segment recommendation may be based at least in part on one or more of: width discrepancies between the upper and lower left posterior segments, width discrepancies between the upper and lower right posterior segments, width discrepancies between the upper and lower jaws; and an initial bite class of opposing canine teeth. For example, the initial bite class or predicted bite class of opposing canine teeth may be one of: class I, class II or class III. In some examples the segment recommendation may be based on one or more of: the presence of missing or extracted canine teeth in the digital model; and a predicted bite class of opposing canine teeth after implementation of the orthodontic treatment plan. The predicted bite class of opposing canine teeth may be determined based on whether one or both of the opposing canine teeth are missing or expected to be extracted during the orthodontic treatment. The predicted bite class of opposing canine teeth may be determined based on whether one or more incisors is expected to be extracted during the orthodontic treatment. In some examples the predicted bite class of opposing canine teeth is determined based on whether the upper and lower anterior segments include a mixed dentition, where the mixed dentition includes a combination of primary and permanent teeth. The predicted bite class of opposing canine teeth may be determined for opposing left canine teeth and opposing right canine teeth.

In any of these methods and apparatuses, the segment recommendation may be based on a degree of Bolton excess of opposing anterior teeth. The segment recommendation may be based on whether the degree of Bolton excess of opposing anterior teeth is greater than a predetermined value. In some examples the segment recommendation is based on whether an overjet is to be maintained after the orthodontic treatment. The segment recommendation may be based on whether a width discrepancy of upper and lower left posterior segments is outside of a first predetermined tolerance, whether a width discrepancy of upper and lower right posterior segments is outside of the first predetermined tolerance, and whether a width discrepancy of the upper and lower jaws is outside a second predetermined tolerance.

In any of the methods and apparatuses (e.g., systems) described herein generating a segment recommendation may include modeling an effect of an interproximal reduction and an interproximal spacing procedure in each of the segments to calculate a benefit estimate. For example, a method may include: receiving a digital model including teeth of an upper jaw and a lower jaw; grouping teeth of the digital model into a plurality of segment, including at least two posterior segments and at least one anterior segment for each of the upper jaw and the lower jaw, wherein each of the posterior segments includes at least one molar and at least one premolar, and each of the anterior segments includes at least one incisor and at least one canine tooth; generating a segment recommendation for each segment of the plurality of segments indicating if one or more of interproximal adjustment procedures should be performed as part of an orthodontic treatment plan, by modeling an effect of an interproximal reduction (IPR) and an interproximal spacing procedure in each of the segments to calculate a benefit estimate based on one or more of: a presence of missing teeth, a presence of primary teeth, a degree of overjet, a bite classification of the teeth, and a degree of Bolton excess of the teeth; and outputting the segment recommendations.

Also described herein are systems configured to perform any of the methods described herein. For example, an orthodontic treatment planning system may include: a computing device comprising a non-transitory computing device readable medium having instructions stored thereon that are executable by a processor to cause the computing device to: receive a digital model including teeth of an upper jaw and a lower jaw; group teeth of the digital model into a plurality of segment, including at least two posterior segments and at least one anterior segment for each of the upper jaw and the lower jaw, wherein each of the posterior segments includes at least one molar and at least one premolar, and each of the anterior segments includes at least one incisor and at least one canine tooth; generate a segment recommendation for each segment of the plurality of segments indicating if one or more of interproximal adjustment procedures should be performed as part of an orthodontic treatment plan, based on one or more of: a presence of missing teeth, a presence of primary teeth, a degree of overjet, a bite classification of the teeth, and a degree of Bolton excess of the teeth; and output the segment recommendations.

Any of these systems may include or be part of an intraoral scanning device. For example, an intraoral scanning device may be configured to direct radiation (including but not limited to visible light, infrared light, x-ray radiation, fluorescence, etc.) from one or more radiation sources toward teeth of an upper jaw and a lower jaw within an oral cavity, the intraoral scanning device including one or more detectors configured to collect images of the teeth.

For example, an orthodontic treatment planning system may include: a computing device comprising a non-transitory computing device readable medium having instructions stored thereon that are executable by a processor to cause the computing device to: receive a digital model including teeth of an upper jaw and a lower jaw; group teeth of the digital model into a plurality of segment, including at least two posterior segments and at least one anterior segment for each of the upper jaw and the lower jaw, wherein each of the posterior segments includes at least one molar and at least one premolar, and each of the anterior segments includes at least one incisor and at least one canine tooth; generate a segment recommendation for each segment of the plurality of segments indicating if one or more of interproximal adjustment procedures should be performed as part of an orthodontic treatment plan, by modeling an effect of an interproximal reduction (IPR) and an interproximal spacing procedure in each of the segments to calculate a benefit estimate based on one or more of: a presence of missing teeth, a presence of primary teeth, a degree of overjet, a bite classification of the teeth, and a degree of Bolton excess of the teeth; and output the segment recommendations.

In some examples a method may include: receiving a three-dimensional (3D) digital model of a patient's teeth, wherein the 3D digital model includes including three-dimensional (3D) representations of teeth on of an upper jaw and a lower jaw of the patient; dividing the upper jaw into a plurality of first segments, wherein each of the plurality of first segments has a plurality of teeth, and the lower jaw into a plurality of second segments, wherein each of the plurality of second segments has a plurality of teeth; determine whether or not to modify tooth spacing on the upper and lower jaws using by applying one or more spacing factors on each of the plurality of first segments and each of the plurality of second segments; using a determination of whether or not to modify tooth spacing on the upper and lower jaws to recommend one or more interproximal space-related procedures on the upper and lower jaws; and providing a recommendation of the one or more interproximal space-related procedures for digital dental treatment planning of the patient's teeth with the 3D digital model.

Any of these method and apparatuses may include performing the one or more interproximal space-related procedures on the upper and lower jaws. Performing may include providing a recommendation of the one or more interproximal space-related procedures as part of a digital dental treatment planning of the patient's teeth with the 3D digital model. In some examples the performance of the one or more interproximal space-related procedures on the upper and lower jaws may be performed digitally, e.g., including showing a digital representation (e.g., 3D virtual model) of the patient's teeth after completion of the recommended one or more interproximal space-related procedures on the upper and lower jaws. In some cases performance of the one or more interproximal space-related procedures on the upper and lower jaws may include modifying the subject's upper and lower jaws as guided by the one or more interproximal space-related procedures.

The determination of whether or not to modify tooth spacing on the upper and lower jaws may include one or more indications whether interproximal spaces are allowed or not on the plurality of first segments or the plurality of second segments. Th determination of whether or not to modify tooth spacing on the upper and lower jaws may include an evaluation of the plurality of first or second segments against one or more prediction trees, wherein the one or more prediction trees comprise a plurality of decision nodes representing clinical criteria for evaluating spacing determinations on the plurality of first or second segments. In some examples, the determination of whether or not to modify tooth spacing on the upper and lower jaws comprises an evaluation of the plurality of first or second segments against one or more prediction trees, wherein the one or more prediction trees comprise a plurality of decision nodes representing clinical criteria for evaluating spacing determinations on the plurality of first or second segments; and the prediction trees comprise one or more of: a posterior IPR prediction tree, a canine class prediction tree, a mixed canine class decision tree, and an anterior IPR spacing prediction tree.

The one or more spacing factors may include one or more clinical setup characteristics for the patient's teeth. In some examples the one or more spacing factors may comprise one or more clinical setup characteristics for the patient's teeth; the one or more clinical setup characteristics comprise one or more of: a presence of missing teeth, a presence of primary teeth, a degree of overjet, a bite classification of the teeth, and a degree of Bolton excess of the teeth. The one or more spacing factors may comprise one or more prescribed settings for the patient's teeth.

The one or more spacing factors may comprise one or more prescribed settings for the patient's teeth; and the one or more prescribed settings comprise one or more of: settings to manage a interproximal reduction (IPR) procedure on the plurality of first segments or the plurality of second segments, settings to manage spaces on the plurality of first segments or the plurality of second segments, a bite class goal for the patient's teeth, and an overjet goal for the patient's teeth.

The plurality of first segments may comprise one or more of: a first posterior segment with a plurality of upper posterior teeth, and a plurality of first anterior segments, each with a plurality of upper anterior teeth; the plurality of second segments comprise one or more of: a second posterior segment with a plurality of lower posterior teeth, and a plurality of second anterior segments, each with a plurality of lower anterior teeth; or some combination thereof.

Any of these methods may include incorporating the recommendation of the one or more interproximal space-related procedures into a final position determination in a digital dental treatment plan. Any of these methods may include using the recommendation of the one or more interproximal space-related procedures to recommend a stage of a digital treatment plan to perform the one or more interproximal space-related procedures.

In some examples the method may include displaying the recommendation of the one or more interproximal space-related procedures on a computer-implemented display; for example, displaying the recommendation of the one or more interproximal space-related procedures on a representation of the 3D digital model of the patient's teeth. In some examples the method may include displaying the recommendation of the one or more interproximal space-related procedures on a representation of the 3D digital model of the patient's teeth; and processing one or more user interactions related to the one or more interproximal space-related procedures with the representation of the 3D digital model of the patient's teeth.

Any of these method may include taking an intraoral scan of the patient's teeth and converting the intraoral scan into the 3D dental model.

The method may include exporting a digital treatment plan comprising the recommendation of the one or more interproximal space-related procedures for design of aligners to implement a digital treatment plan.

Also described herein are systems including one or more processors; and memory coupled to the one or more processors storing computer-program instructions that, when executed by the one or more processors, cause the system to perform: receiving a three-dimensional (3D) digital model of a patient's teeth, wherein the 3D digital model includes including three-dimensional (3D) representations of teeth on of an upper jaw and a lower jaw of the patient; dividing the upper jaw into a plurality of first segments, wherein each of the plurality of first segments has a plurality of teeth, and the lower jaw into a plurality of second segments, wherein each of the plurality of second segments has a plurality of teeth; determine whether or not to modify tooth spacing on the upper and lower jaws using by applying one or more spacing factors on each of the plurality of first segments and each of the plurality of second segments; and using a determination of whether or not to modify tooth spacing on the upper and lower jaws to recommend one or more interproximal space-related procedures on the upper and lower jaws; providing a recommendation of the one or more interproximal space-related procedures for digital dental treatment planning of the patient's teeth with the 3D digital model.

Any of these systems may include an output (e.g., display) coupled to the one or more processors and operative to display the recommendation of the one or more interproximal space-related procedures. For example, any of these systems may include a display coupled to the one or more processors and operative to display the recommendation of the one or more interproximal space-related procedures on a representation of the 3D digital model of the patient's teeth. As mentioned, any of these systems may include an intraoral scanner configured to take a scan of the patient's teeth. For example, any of these systems may include an intraoral scanner configured to take a scan of the patient's teeth, wherein the computer-program instructions, when executed by the one or more processors, cause the system to convert the intraoral scan into the 3D dental model.

These and other aspects are described herein. All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A illustrates an example side view of a dentition showing an overjet.

FIG. 1B illustrates an example side view of a dentition showing relative cusp locations for determining bite classes for molar and canine teeth.

FIG. 1C illustrates an example expanded presentation of a set of upper and lower teeth showing aspects of a Bolton analysis.

FIG. 2 illustrates an example decision tree for predicting whether interproximal reduction (IPR) would be beneficial for a particular side of a posterior segment of a set of teeth in an orthodontic treatment plan.

FIGS. 3A-3H illustrate example decision trees for predicting whether IPR or an interproximal spacing procedure would be beneficial for an anterior segment of a set of teeth in an orthodontic treatment plan.

FIGS. 4A and 4B illustrate charts showing example data from a study comparing metrics related to treatment plans generated using the prediction models described herein to previous treatment planning methods.

FIG. 5A illustrates an example of a method as described herein.

FIG. 5B illustrates an example of a method as described herein.

FIG. 5C illustrates an example of a method as described herein.

FIG. 6 is a diagram illustrating an example interproximal adjustment prediction tool.

FIG. 7 is a diagram illustrating an example computing device for predicting whether an interproximal adjustment procedure would be beneficial as part of an orthodontic treatment plan.

FIG. 8 shows a diagram illustrating an example of a computing environment including the interproximal adjustment prediction module(s), described herein.

### DETAILED DESCRIPTION

The apparatuses, systems, and methods described herein relate to interproximal adjustment procedures, such interproximal reduction (IPR) and interproximal spacing procedures. IPR and interproximal spacing procedures are typically used in orthodontics to correct a patient's bite malocclusions. For example, relatively large teeth may not allow adjacent teeth to come close enough together to correctly align corresponding upper and lower teeth. In such cases, an IPR procedure, where some enamel from one or both of adjacent teeth is removed, allows the teeth to come into contact with each other without overcrowding. In some cases, the teeth on a first jaw (e.g., upper jaw) may be relatively small compared to opposing teeth on an opposite second jaw (e.g., lower jaw), such that positioning the teeth in a proper bite configuration creates too much space between the smaller teeth of the first jaw. In such cases, the interproximal space between the smaller teeth of the first jaw may be intentionally planned to provide room for the addition of a tooth-like material (e.g., composite, crown and/or veneer) to one or both of the adjacent smaller teeth to fill the space.

IPR and interproximal spacing procedures may be implemented as part of orthodontic treatment plans that use a series of removable dental appliances, such as dental aligners. The treatment plans include multiple treatment stages in which the teeth are incrementally repositioned from an initial tooth arrangement toward a final tooth arrangement. Typically, one dental appliance (e.g., aligner) is associated with each stage. For example, a treatment plan that includes 25 stages may involve the use of 25 dental appliances. A dental appliance associated with a particular stage of the treatment plan is configured to move the teeth from a first arrangement at the beginning of the particular stage toward a second arrangement at the end of the particular stage.

The treatment plan may be represented by one or more digital three-dimensional (3D) models of the patient's dentition. Typically, an initial 3D model of the patient's dentition is obtained by scanning the patient's dentition using a dental scanner. A final 3D model representing a desired configuration of the patient's teeth may be determined based on applying repositioning forces on one or more teeth of the initial 3D model. Each stage of the treatment plan may be associated with a first intermediate 3D model representing the dentition at the beginning of the treatment stage and second intermediate 3D model representing the dentition at the end of the treatment stage. In some cases, the repositioning forces may be digitally simulated on a 3D model of a dentition in determining proper forces to be applied to the teeth at various stages of the treatment plan.

The dental appliances described herein include dental aligners (“aligners”) that are configured to fit on the patient dentition and be removable by the patient. The individual aligners may be made of a polymeric shell and shaped to have a teeth-receiving cavity for receiving the teeth of a dental arch. The teeth-receiving cavity may have a geometry corresponding to an end tooth arrangement intended for that stage of the treatment plan. That is, when an aligner is first worn by the subject, certain teeth will be misaligned relative to an undeformed geometry of the aligner cavity. The aligner, however, is sufficiently resilient to accommodate or conform to the misaligned teeth, and will resiliently apply sufficient force against such misaligned teeth to reposition the teeth toward the end arrangement desired for that treatment stage.

In some cases, an aligner may be configured to apply the repositioning forces on the teeth with one or more dental attachments that are temporarily bonded to the patient's teeth. Attachments are small ridges that are often tooth-colored and made of orthodontic material designed to blend with the tooth enamel. Attachments may have different shapes and sizes and be made of different materials, depending on their function. The aligner may be configured to provide directed pressure against the attachments, making more complex directional tooth movement possible. The aligner may have indentations with shapes matching those of the corresponding attachment(s) such that the aligner can apply the prescribed force(s) and fit snugly and smoothly over the attachment(s).

In some cases, digital 3D model versions of the aligners may be created and used as a basis for forming the actual (physical) aligners. The digital aligners may be shaped according to the actual aligners and be configured to fit on the digital models of the dentition. In some cases, the aligners may be formed using a direct fabrication process (e.g., additive manufacturing) where the actual aligners are fabricated based on computer-aided design (CAD) data. In other cases, 3D dentition molds are formed and polymer material (e.g., polymer sheets) are molded onto the 3D dentition molds to form the actual aligner. In some cases, a combination of additive manufacturing, molding, and other manufacturing processes (e.g., polishing and laser cutting) are used.

Integrating an interproximal adjustment procedure into an orthodontic treatment plan may involve adding repositioning forces to teeth in anticipation of the interproximal adjustment procedure. For example, if an IPR procedure is part of the treatment plan, the repositioning forces applied by the aligner(s) may include those that move adjacent teeth closer together than would be allowed if an IPR was not part of the treatment plan. Likewise, if an interproximal spacing procedure is part of the treatment plan, the repositioning forces applied by the aligner(s) may include those that move adjacent teeth farther apart than would be allowed if an interproximal spacing procedure was not part of the treatment plan.

Although interproximal adjustment procedures may be helpful in improving a patient's bite configuration, they are not always necessary or desirable, especially if dental appliances can sufficiently improve the patient's bite configuration without using an interproximal adjustment procedure. At one extreme, a treatment plan may include one or more interproximal adjustment procedures to address even minor bite malocclusions when such malocclusions may be addressed using the dental appliances alone, which may overly complicate the treatment process and/or extend the overall treatment time. At the other extreme, a treatment plan may not include any interproximal adjustment procedures even when necessary, resulting in a final dentition having unacceptable bite malocclusions. Different treatment plans having different tooth repositioning paths may accomplish the same or similar final teeth arrangements. It can be difficult to determine whether an interproximal adjustment procedure would be advantageous for a particular patient's dentition. The systems described herein can accurately predict whether one or more interproximal adjustment procedures would be effective in treating bite malocclusions and/or would be conducive to treatment goals.

The prediction of whether an interproximal adjustment may be beneficial can be made based, in part, on clinically derived characteristics of the patient's “initial” dental condition prior to initiation of an orthodontic treatment, reinitiating of an orthodontic treatment, or between stages of an orthodontic treatment plan (e.g., when the treatment plan is changed mid-course). The teeth of the upper and lower jaws into posterior and anterior regions (also referred to as sections or segments). Each of the posterior segments may include molars and premolars, and each of the anterior segments may include incisors and canine teeth. For example, posterior segments may include an upper left posterior segment (left side of the upper jaw), an upper right posterior segment (right side of the upper jaw), a lower left posterior segment (left side of the lower jaw), and a lower right posterior segment (right side of the lower jaw). The anterior segments may include an upper anterior segment (of the upper jaw) and a lower anterior segment (of the lower jaw).

The initial dental condition may include one or more clinically characterized malocclusions. In some cases, the determination may be made based on one or more of: a bite class (e.g., of molars and/or canines); the existence of any missing or extracted teeth (e.g., canine, bicuspid, incisor, molar); the presence and severity of a Bolton discrepancy; and the presence and type of overjet (e.g., normal, positive or negative). In some examples, the determination may further be based on prescribed settings chosen by the user (e.g., clinician). Such prescribed settings may include one or more of: whether IPR is allowed in the dentition segment; whether interproximal spaces are allowed in the dentition; bite class goal(s) (e.g., improve bite class or maintain bite class) and/or whether to show a resulting bite class; and overjet goal(s) (e.g., improve overjet or maintain overjet).

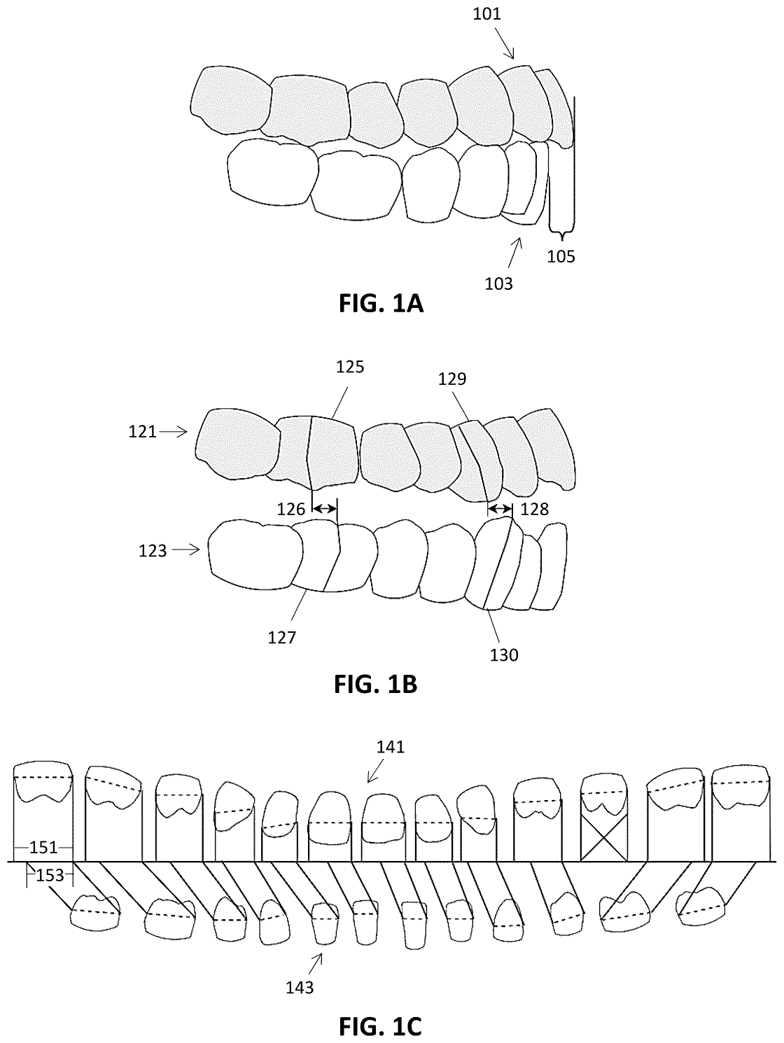

FIGS. 1A-1C illustrate three example clinical factors that may be used to predict whether interproximal adjustments (IPR and/or interproximal spacing) would be beneficial. FIG. 1A shows an example dentition illustrating an overjet where front upper teeth 101 stick out past the front lower teeth 103. The degree of overjet can be characterized by a horizonal distance 105 that the front upper teeth 101 extend past the corresponding front lower teeth 103 by a threshold amount or greater. In addition, the type of overjet may be established. For example, a normal overjet (overbite) is when the front upper teeth 101 stick out past the front lower teeth 103 by the threshold amount or greater, whereas a negative overjet (underbite) is when the front lower teeth 103 stick out past the front upper teeth 101.

FIG. 1B shows an example set of upper teeth 101 and lower teeth 103 illustrating examples of bite classes. In general, bite class refers to how the upper teeth 101 and the lower teeth 103 meet during bite occlusion. Bite classes range from class I to class III, with class I bites considered normal. For a normal bite occlusion, the cusp of an upper first molar 125 should rest in the corresponding groove of a lower first molar 127. The bite class of the upper first molar 125 and the lower first molar 127 may be characterized by measuring a horizonal distance 126 between the cusp of the upper first molar 125 and the corresponding groove of the lower first molar 127. The bite class of an upper canine 129 and a corresponding lower canine 130 may be characterized by measuring a horizonal distance 128 between the cusp of the upper canine 129 and the cusp of the lower canine 130.

FIG. 1C shows an example expanded presentation of corresponding upper (maxillary) teeth 141 and lower (mandibular) teeth 143 illustrating aspects of a Bolton analysis. In the example shown, there is a missing lower premolar. A Bolton analysis can be used to determine discrepancies between the size of upper 141 and lower 143 teeth and may be used to determine an optimum inter-arch relationship. An overall Bolton analysis may involve measuring and comparing the mesio-distal widths (e.g., 151) of all maxillary teeth to the mesio-distal widths (e.g., 153) of all corresponding mandibular teeth to provide a Bolton ratio for the entire arch. In some cases, an anterior Bolton analysis compares the sum of mesio-distal widths of the anterior six (incisors and canines) mandibular teeth to the sum of mesio-distal widths of the anterior six (incisors and canines) maxillary teeth, providing a Bolton ratio for the anterior teeth. The anterior Bolton ratio may be most often used because there is generally more variability in anterior tooth size than posterior tooth size (within an individual), and because patients generally care more about crowding and spacing in the anterior. A Bolton discrepancy (overall or anterior) refers to the differences in size that occur between the teeth of the upper and lower jaw. A Bolton excess is a particular type of Bolton discrepancy, and refers to when the anterior maxillary teeth have relatively large mesio-distal widths compared to the mesio-distal widths of the anterior mandibular teeth. Above a certain degree of Bolton discrepancy (e.g., Bolton excess), it may no longer be possible to position the teeth in a normal bite without IPR or providing a space between adjacent teeth, as described above.

Other clinical factors that may be considered include whether there are any missing teeth (e.g., deciduous teeth and/or extracted teeth), and if so, which teeth are missing (e.g., canine, bicuspid, incisor, premolar and/or molar).

Once clinical factors related to malocclusions (if any) at different regions of a patient's teeth have been established, one or more computer analyses may be used to predict whether one or more interproximal adjustments (IPR and/or interproximal spacing) would be beneficial to include in an orthodontic treatment plan. Example computer analyses may include any of a number of analyses techniques, such as regression analysis(es), decision tree analysis(es) and/or machine learning (e.g., ensemble learning, support vector machine, etc.). For machine learning analysis(es), the training data set may include using data associated with the teeth of a number of different patients. In some cases, the training data set may be filtered to assure a good training data set. In some instances, the training data may be automatically filtered based on predetermined factors. In some instances, a dental professional (e.g., orthodontist, dentist, etc.) may be involved in filtering the training data.

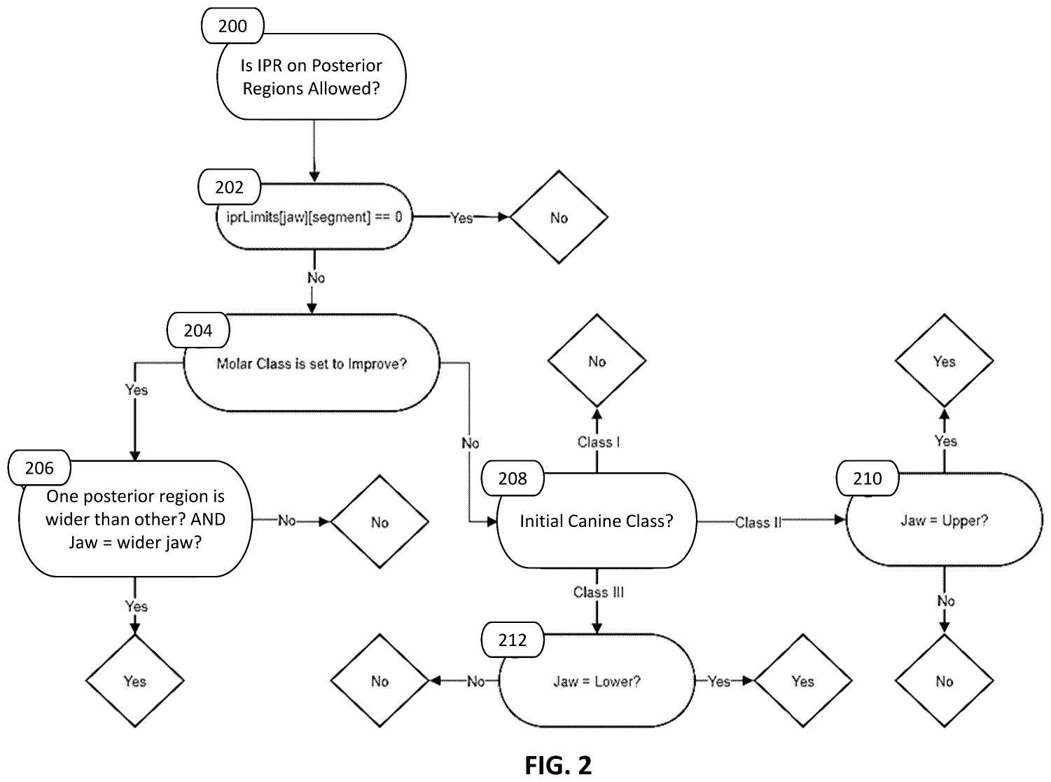

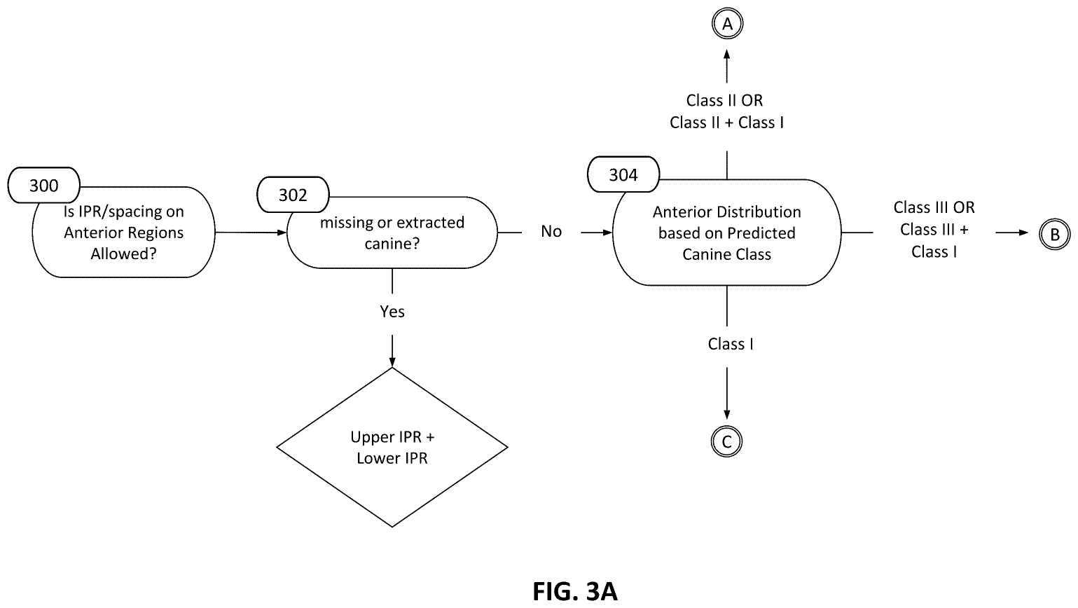

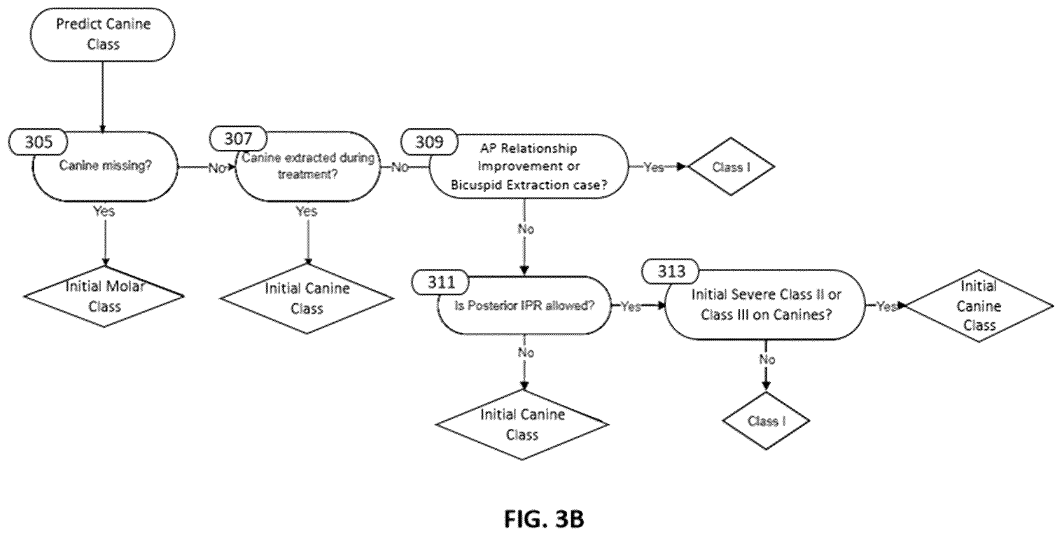

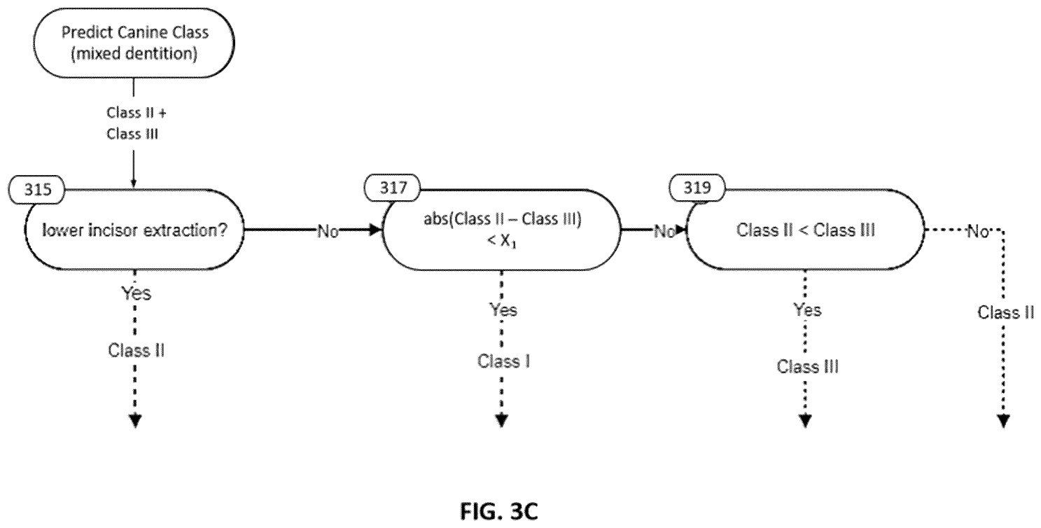

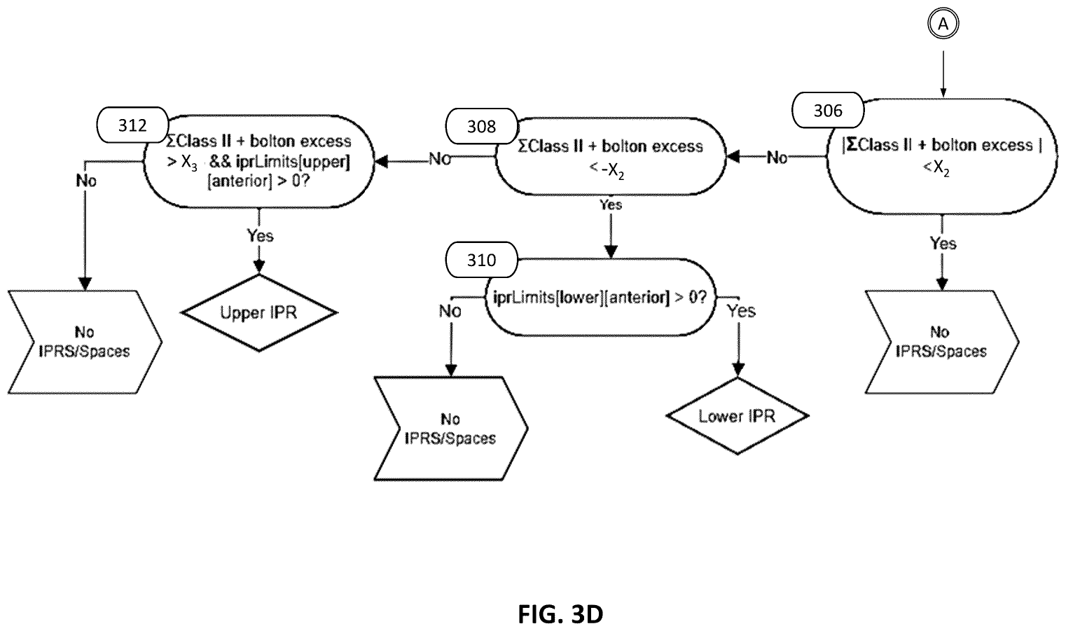

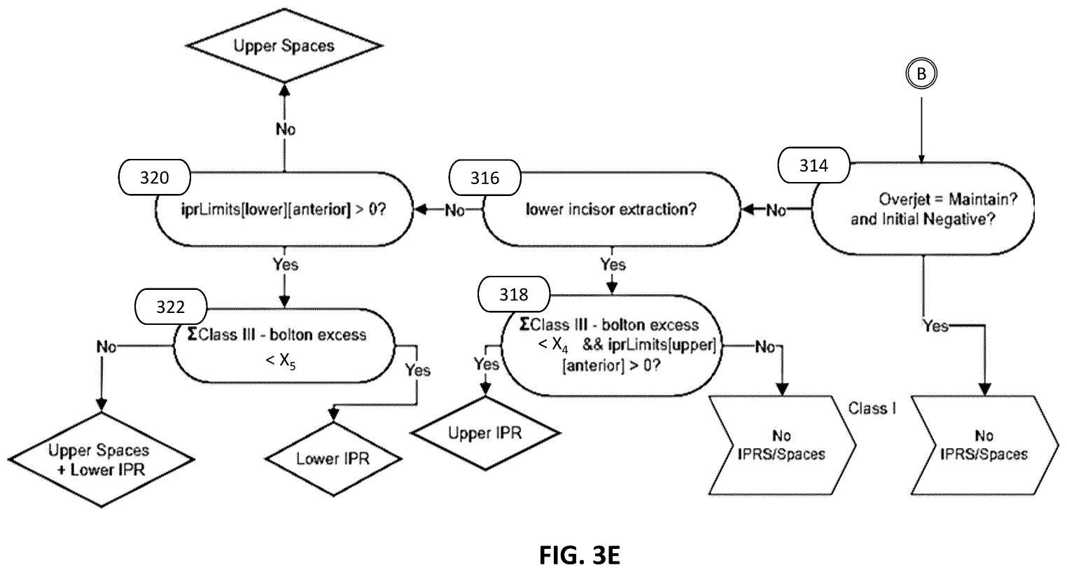

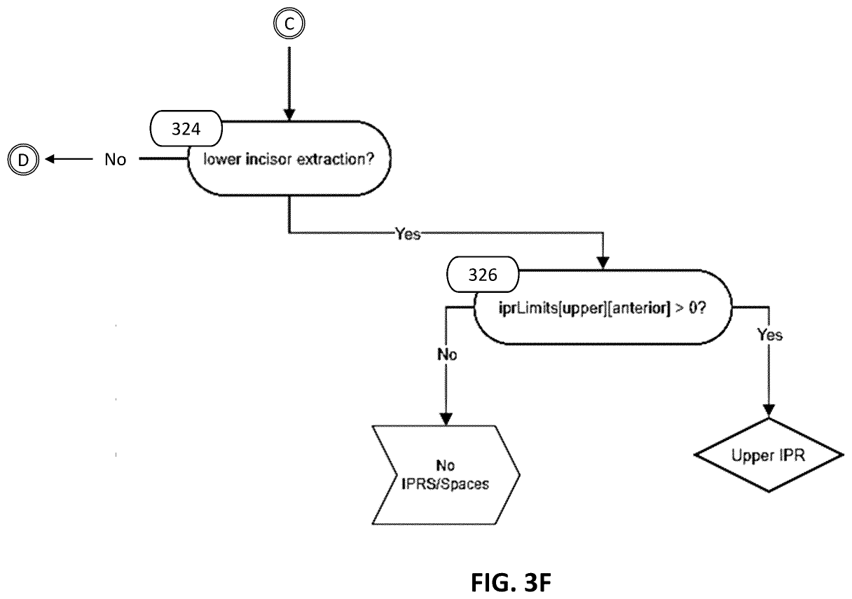

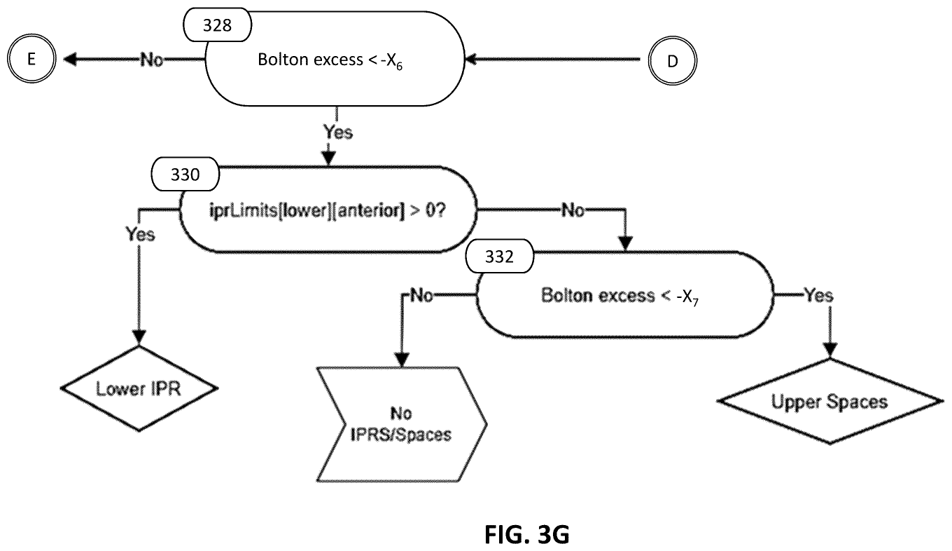

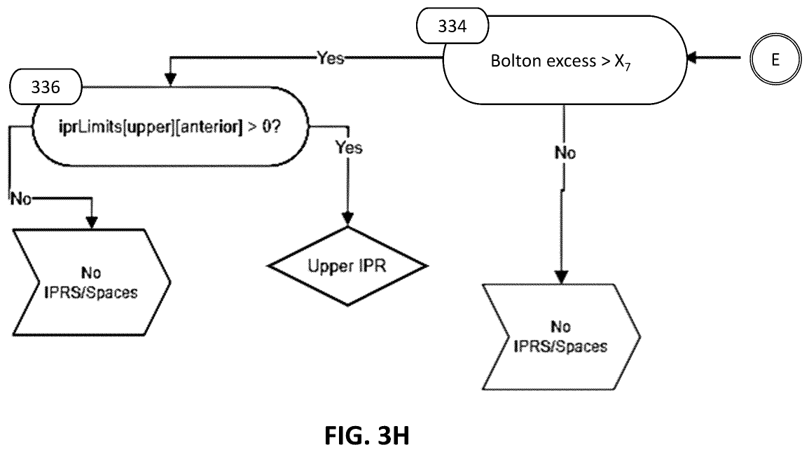

In some examples, an array of decision trees (also referred to as prediction trees) may be used to predict whether IPR and/or interproximal spacing would be beneficial in a treatment plan. The array of prediction trees may include one or more decision trees for each of the posterior segments of the teeth and one or more different decision trees for each of the anterior segments of the teeth. FIGS. 2-3H show example decision trees for posterior and anterior segments according to some embodiments.

FIG. 2 shows an example decision tree for predicting whether IPR would be beneficial for a particular side (e.g., left or right) of a posterior segment (upper posterior segment or lower left posterior segment) of teeth. At 200 a determination is made as to whether IPR is allowed in the particular posterior segment. This determination may be made based on a 3D model of a patient's teeth (e.g., formed from data associated with one or more intraoral scans). This determination may be entered and received by a user (e.g., dental professional). Alternatively, the computer may include instructions for determining whether IPR is (or should be) allowed without input from a user. At 202, it is determined whether the arrangement of teeth within the particular segment is outside limits of where IPR can be performed. If the arrangement of teeth is outside the limits (“Yes”), then the prediction result is not to perform a posterior IPR (“No”). If the arrangement of teeth is not outside the limits (“No”), at 204 it is determined whether settings allow for Molar Class improvement.

If the settings allow for Molar Class improvement (“Yes”), at 206 a determination is made as to whether one posterior segment (e.g., lower left, lower right, upper left or upper right) is wider than the opposing posterior segment (e.g., upper left, upper right, lower left or lower right) outside of a first predetermined tolerance AND whether the upper and lower jaws have different widths outside of a second predetermined tolerance. In some examples, the widths of the posterior segments may be measured from the most posterior tooth to the most anterior tooth of matching posterior segments (e.g., lower left and upper left, or lower right and upper right). The width of the upper and lower jaws may be measured from molar to opposing molar. For example, for the upper jaw, the jaw width may be measured from a molar on the right side to a corresponding molar on the left side across the patient's palate. In some cases, an average width discrepancy for all molars and/or premolars is used. In some cases, the first and second predetermined tolerances are the same. In some examples, the first and/or second predetermined tolerance ranges between about 0.4 mm and 0.5 mm (e.g., 0.4 mm, 0.42 mm, 0.45 mm, 0.48 mm or 0.5 mm).

If it is determined that one posterior segment is wider than the other posterior segment by the first predetermined tolerance AND the teeth arrangement of one jaw is wider than the other jaw by the second predetermined tolerance (“YES”), then the prediction result is to perform one or more posterior IPRs (on posterior teeth). If it is determined that one segment is wider than the other segment AND the teeth arrangement of one jaw is wider than the other jaw within the predetermined tolerance (“No”), then the prediction result is not to perform a posterior IPR.

Returning to 204, if the settings do not allow for Molar Class improvement (“No”), at 208 an initial canine bite class is determined. The initial canine bite class may be determined based on the positions of the patient's teeth prior to treatment or prior to a particular stage of a treatment (e.g., mid-course). The initial canine bite class may be classified as class I, class II or class III based on the severity of malocclusion. If the initial canine bite class is characterized as being class I, then the prediction result is not to perform a posterior IPR (“No”). If the initial canine bite class is characterized as being class II, at 210 a determination is made as to whether the canine malocclusion is on the upper jaw. If the canine malocclusion is on the upper jaw, then the prediction result is to perform one or more posterior IPRs (“YES”). If the canine malocclusion is not on the upper jaw, then the prediction result is not to perform a posterior IPR (“No”). Returning to 208, if the initial canine bite class is characterized as being class III, at 212 a determination is made as to whether the canine malocclusion is on the lower jaw. If the canine malocclusion is on the lower jaw, then the prediction result is to perform one or more posterior IPRs (“YES”). If the canine malocclusion is not on the lower jaw, then the prediction result is not to perform a posterior IPR (“No”).

Once the decision tree of FIG. 2 is used to predict whether posterior IPR would be beneficial for a particular posterior segment of teeth, the same process may be used to predict whether posterior IPR would be beneficial for other posterior segments of the teeth (e.g., upper left posterior segment, upper right posterior segment, lower left posterior segment, or lower right posterior segment).

FIGS. 3A-3H show example decision trees for predicting whether interproximal adjustment (IPR and/or interproximal spacing) would be beneficial for a particular anterior segment of teeth (upper anterior segment or lower anterior segment). In FIG. 3A, a determination is made 300 as to whether interproximal adjustment(s) (e.g., IPR and/or interproximal spacing) is allowed in the particular anterior segment. This determination may made based on a 3D model of a patient's teeth (e.g., formed from data associated with one or more intraoral scans). This determination may be entered and received by a user (e.g., dental professional). Alternatively, the computer may include instructions for determining whether interproximal adjustment(s) is (or should be) allowed without input from a user. At 302 a determination is made as to whether there is a missing and/or extracted canine. If there is a missing and/or extracted canine (“Yes”), then the prediction result is to perform an anterior IPR. If there is not a missing or extracted canine (“No”), at 304 an anterior distribution of the teeth based on predicted canine class is made. The predicted canine class may correspond to the predicted bite class of opposing canine teeth (on each side-left and/or right) after implementation of the orthodontic treatment. The predicted canine class may depend on factors related to the initial dentition (e.g., prior to orthodontic treatment) and during treatment of the teeth. An example decision tree for determining a predicted canine bite class is described in detail with reference to FIG. 3B, and an example decision tree for determining a predicted canine bite class for a mixed dentition is described in detail with reference to FIG. 3C. If the anterior distribution of teeth based on predicted canine class is class II or class II and class I (“A”), further analysis is performed according to FIG. 3D. If the anterior distribution of teeth based on predicted canine class is class III or class III and class I (“B”), further analysis is performed according to FIG. 3E. If the anterior distribution of teeth based on predicted canine class is class I (“C”), further analysis is performed according to FIG. 3F.

FIG. 3B shows an example decision tree for predicting a canine bite class for a particular side (e.g., left or right) of the jaw. At 305, a determination is made as to whether there is a missing canine (e.g., upper or lower) of the particular side of the jaw in the initial dentition. If a canine is missing (“Yes”), the initial molar bite class is used in the prediction. The initial molar bite class may be determined based on the positions of the patient's teeth prior to treatment or prior to a particular stage of a treatment (e.g., mid-course). If a canine is not missing (“No”), at 307 a determination as to whether a canine is to be extracted during treatment. If a canine is to be extracted during treatment (“Yes”), the initial (or current) canine bite class is used in the prediction. If a canine is not to be extracted during treatment (“No”), at 309 a determination is made as to whether the case involves an anterior-posterior (AP) relationship improvement or a bicuspid extraction. If the case involves an AP relationship improvement or a bicuspid extraction (“Yes”), a canine bite class I is used in the prediction. If the case does not involve an AP relationship improvement or a bicuspid extraction (“No”), at 311 a determination is made as to whether a posterior IPR is allowed (e.g., as determined by a professional and/or is possible). If a posterior IPR is not allowed (“No”), the initial (or current) canine bite class is used in the prediction. If a posterior IPR is allowed (“Yes”), at 313 a determination is made as to whether the initial canine bite class is a severe class II or class III case. If the initial canine bite class is not a severe class II or class III case (“No”), a canine bite class I is used in the prediction. If the initial canine bite class is a severe class II or class III case (“Yes”), the initial (or current) canine bite class is used in the prediction.

FIG. 3C shows an example decision tree for resolving a canine bite class for a mixed dentition at a particular side (e.g., left or right) of the jaw. In general, a mixed dentition includes a combination of primary and permanent teeth. If the initial canine bite class is class II or III, at 315 a determination is made as to whether there will be a lower incisor extraction during treatment. If there will be a lower incisor extraction during treatment (“Yes”), a canine bite class II is used in the prediction. If there will not be a lower incisor extraction during treatment (“No”), at 317 a determination is made as to whether an absolute value of the number of class II teeth minus the number of class III teeth is less than a first predetermined value “X₁”. In some cases, the first predetermined value “X₁” ranges from about 0.35 mm and 0.55 mm (e.g., 0.35 mm, 0.4 mm, 0.45 mm, 0.47 mm, 0.5 mm, or 0.55 mm). If the abs (class II-class III) is less than the first predetermined value (“Yes”), a canine bite class I is used in the prediction. If the abs (class II-class III) is not less than the first predetermined value (“No”), at 319 a determination is made as to whether the number of class II teeth is less than the number of class III teeth. If the number of class II teeth is less than the number of class III teeth (“Yes”), a canine bite class III is used in the prediction. If the number of class II teeth is not less than the number of class III teeth (“No”), a canine bite class II is used in the prediction.

FIG. 3D shows an example decision tree continuing from “A” in the decision tree of FIG. 3A. The decision tree of FIG. 3D is for predicting whether one or more interproximal adjustments (e.g., IPR and/or spacing) would be beneficial when the anterior distribution is predicted to be class II or a combination of class II and class I. At 306 a determination is made as to whether the absolute value of a summation of the number of class II and Bolton excess is less than a second predetermined value “X₂”. In some examples, the second predetermined value “X₂” ranges from about 0.45 mm to about 0.6 mm (e.g., 0.45 mm, 0.47 mm, 0.5 mm, 0.52 mm, 0.55 mm or 0.6 mm). If so (“Yes”), then the prediction result is not to perform an anterior IPR or anterior interproximal spacing procedure as part of a treatment plan. If not (“No”), at 308 a determination is made as to whether a summation of class II teeth and Bolton excess is less than the negative of the second predetermined value “X₂”. If so (“Yes”), at 310 a determination is made as to whether IPR limits of the lower anterior teeth are greater than zero. If so (“Yes”), then the prediction result is to perform a lower anterior IPR procedure as part of a treatment plan. If not (“No”), then the prediction result is not to perform an anterior IPR or anterior interproximal spacing procedure as part of a treatment plan. Returning to 308, if the determination is made that the summation of class II teeth and Bolton excess is not less than the negative of the second predetermined value “X₂”, at 312 a determination is made as to whether a summation of class II teeth and Bolton excess is greater than a third predetermined value “X₃” and whether upper anterior IPR limits are greater than zero. In some examples, the third predetermined value “X₃” ranges from about 0.6 mm to about 0.9 mm (e.g., 0.6 mm, 0.65 mm, 0.7 mm, 0.75 mm, 0.8 mm, 0.85 mm or 0.9 mm). If so (“Yes”), then the prediction result is to perform an upper anterior IPR procedure as part of a treatment plan. If not (“No”), then the prediction result is not to perform an anterior IPR or anterior interproximal spacing procedure as part of a treatment plan

FIG. 3E shows an example decision tree continuing from “B” in the decision tree of FIG. 3A. The decision tree of FIG. 3E is for predicting whether one or more interproximal adjustments (e.g., IPR and/or spacing) would be beneficial when the anterior distribution is predicted to be class III or a combination of class III and class I. At 314 a determination is made as to whether a prescription (e.g., from a dental professional) requires an overjet to be maintained and the initial dental model includes a negative overjet. If so (“Yes”), then the prediction result is not to perform an anterior IPR or anterior interproximal spacing procedure as part of a treatment plan. If not (“No”), at 316 a determination is made as to whether there will be any lower incisor extractions. If so (“Yes”), at 318 a determination is made as to whether a summation of class III teeth minus Bolton excess is less than a fourth predetermined “X₄” and lower anterior IPR limits are greater than zero. In some examples, the fourth predetermined value “X₄” ranges from about 4 mm to about 6 mm (e.g., 4 mm, 4.5 mm, 5 mm, 5.5 mm or 6 mm). If not (“No”), then the prediction result is not to perform an anterior IPR or anterior interproximal spacing procedure as part of a treatment plan. If so (“Yes”), then the prediction result is to perform a lower anterior IPR procedure as part of a treatment plan. Returning to 316, if it is determined that there will not be a lower incisor extraction (“No”), at 320 a determination is made as to whether lower anterior IPR limits are greater than zero. If not (“No”), then the prediction result is to perform an upper anterior interproximal spacing procedure as part of a treatment plan. If so (“Yes”), at 322 a decision is made as to whether a summation of class III teeth minus Bolton excess is less than a fifth predetermined “X”. In some examples, the fourth predetermined value “X₄” ranges from about 2 mm to about 4 mm (e.g., 2 mm, 2.5 mm, 2.7 mm, 3 mm, 3.7 mm or 4 mm). If so (“Yes”), then the prediction result is to perform a lower anterior IPR procedure as part of a treatment plan. If not (“No”), then the prediction result is to perform an upper anterior interproximal spacing and a lower anterior IPR procedures as part of a treatment plan.

FIG. 3F shows an example decision tree continuing from “C” in the decision tree of FIG. 3A. The decision tree of FIG. 3F is for predicting whether one or more interproximal adjustments (e.g., IPR and/or spacing) would be beneficial when the anterior distribution is predicted to be class I. At 324 a determination is made as to whether there will be any lower incisor extractions. If so (“Yes”), at 326 a decision is made as to whether upper anterior IPR limits are greater than zero. If so (“Yes”), then the prediction result is to perform an upper anterior IPR procedure as part of a treatment plan. If not (“No”), then the prediction result is not to perform an anterior IPR or anterior interproximal spacing procedure as part of a treatment plan. Returning to 324, if a determination is made that there will not be any lower incisor extractions (“D”), further analysis is performed according to FIG. 3G.

FIG. 3G shows an example decision tree continuing from “D” in the decision tree of FIG. 3F. At 328 a determination is made as to whether the Bolton excess is less than a negative of a sixth predetermined value “X₆”. In some examples, the sixth predetermined value “X₆” ranges from about 0.2 mm to about 0.8 mm (e.g., 0.2 mm, 0.5 mm, 0.7 mm or 0.8 mm). If so (“Yes”), at 330 a decision is made as to whether lower anterior IPR limits are greater than zero. If so (“Yes”), then the prediction result is to perform a lower anterior IPR procedure as part of a treatment plan. If not (“No”), at 332 a decision is made as to whether the Bolton excess is less than a negative of a seventh predetermined value “X₇”. In some examples, the seventh predetermined value“X₇” ranges from about 0.8 mm to about 1.5 mm (e.g., 0.8 mm, 1 mm, 1.2 mm or 1.5 mm). If so (“Yes”), then the prediction result is to perform an anterior interproximal spacing procedure as part of a treatment plan. Returning to 328, if the Bolton excess is not less than a negative of the seventh predetermined value “X₇”, further analysis is performed according to FIG. 3H.

FIG. 3H shows an example decision tree continuing from “E” in the decision tree of FIG. 3G. At 334, a decision is made as to whether the Bolton excess is greater than the seventh predetermined value “X₇”. If not (“No”), then the prediction result is not to perform an anterior IPR or anterior interproximal spacing procedure as part of a treatment plan. If so (“Yes”), at 336 a decision is made as to whether upper anterior IPR limits is greater than zero. If so (“Yes”), then the prediction result is to perform an upper anterior IPR procedure as part of a treatment plan. If not (“No”), then the prediction result is not to perform an anterior IPR or anterior interproximal spacing procedure as part of a treatment plan.

As discussed, the methods described herein may be used to accurately predict whether an interproximal adjustment procedure may be effective in treating bite malocclusions and/or would be conducive to treatment goals. In previous approaches, the dental professional must choose to allow the use of IPR in the treatment plan, and the treatment planning software calculates and optimizes a treatment path accordingly. Unfortunately, this sometimes results in treatment plans that include IPR to make only minor improvements or to improve lesser treatment goals. The methods described herein provide a way for the user (e.g., treatment professional) to predict whether any interproximal adjustment procedure would be beneficial beforehand, thereby providing better control over the treatment plan generation. In practice, this may reduce the incorporation of unnecessary interproximal adjustment procedures in treatment plans without sacrificing clinical goals of the treatment plans.

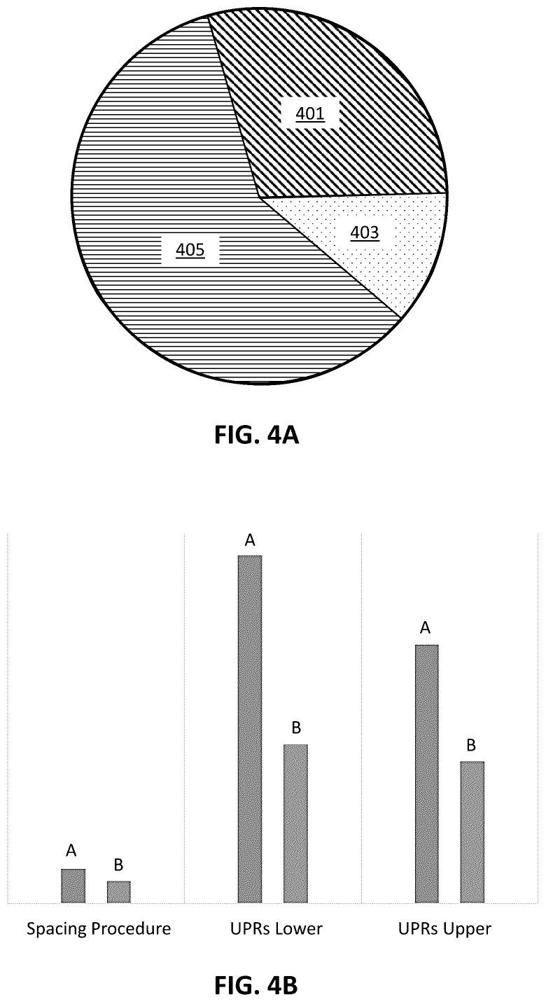

FIGS. 4A and 4B show example data from a study comparing metrics related to the number and extent of interproximal adjustment procedures in treatment plans generated using the prediction models described herein to previous treatment planning methods. The data (measured in millimeters (mm)) is gathered from 330 cases: 110 random personal protocol cases (“IPL” cases), 110 random all-improve cases “All-Improve” cases), and 110 random prescription flow cases (“Rx case”). The study included performing blind tests.

FIG. 4A shows a pie chart indicating the total change in IPRs and interproximal spacing procedures in treatment plans generated using the methods described herein (new approach) compared to previous methods (previous approach). Section 401 represents the percentage of cases where the number of IPRs and interproximal spacing procedures in treatment plans are the same in both the new and previous approaches, which is 29% of the total. Section 403 represents the percentage of cases where the new approach resulted in treatment plans with a greater number of IPRs and interproximal spacing procedures compared to the new approach, which is 11% of the total. Section 405 represents the percentage of cases where the new approach resulted in treatment plans with a lesser number of IPRs and interproximal spacing procedures compared to the previous approach, which is 60% of the total. These results indicate that, in the majority of cases, the new approach resulted in treatment plans with a lesser number of IPRs and interproximal spacing procedures. The overall quality of the treatment plans using the new approach were not worse than the treatment plans using the previous approach, based on validation results.

FIG. 4B shows a bar graph drilling down into the types and location of the interproximal adjustment procedures. “A” represents the number of treatment plans generated using the previous approach, and “B” represents the number of treatment plans generated using the new approach. Results indicate that the new approach resulted in 35% less residual spacing procedures, 55% less lower jaw IPRs, 45% less upper jaw IPRs, 50% less average IPR (mm per case), 20% less average spacing procedures (mm per case), and 1.5 times more jaws without IPRs.

Tables 1 and 2 below show further results from the study.

TABLE 1

| Metric | Before | After |
|---|---|---|
| Ave IPR mm | −0.41 | −0.42 |
| Ave IPR mm per case | −2.47 | −1.25 |
| Ave space mm | 1.41 | 1.73 |
| Ave pace mm per case | 0.48 | 0.38 |
| Cases with any IPR | 250 | 192 |
| Cases with any spaces | 58 | 49 |
| Jaws without IPR | 280 | 437 |

TABLE 2

| | IPL | IPL | All-Improve | All-Improve | Rx | Rx |
|---|---|---|---|---|---|---|
| Case Types | cases | cases | cases | cases | cases | cases |
| Metrics | before | after | before | after | before | after |
| Ave IPR mm per case | −3.21 | −1.23 | −3.39 | −1.8 | −1.61 | −0.92 |
| Ave space mm per case | 0.96 | 0.91 | 0.16 | 0.01 | 0.47 | 0.39 |
| Cases with any IPR | 60 | 43 | 97 | 76 | 93 | 73 |
| Cases with any space | 12 | 16 | 11 | 2 | 35 | 31 |
| Jaws without IPR | 43 | 87 | 36 | 106 | 201 | 244 |
| Ave IPR mm | −0.41 | −0.41 | −0.43 | −0.45 | −0.39 | −0.38 |
| Ave space mm | 2.69 | 2.44 | 0.72 | 0.46 | 1.18 | 1.42 |

In summary, the results of Tables 1 and 2 indicate that compared to the previous approach, the new approach resulted in 2.5 times less IPRs on IPL cases (average IPR, mm per case), 2 times less IPRs on All-Improve cases, 1.5 times less IPRs on Rx cases, 3 times more jaws without IPRs on IPL cases, 2 times more jaws without IPRs on All-Improve cases, and 1.2 times more jaws without IPRs on Rx cases.

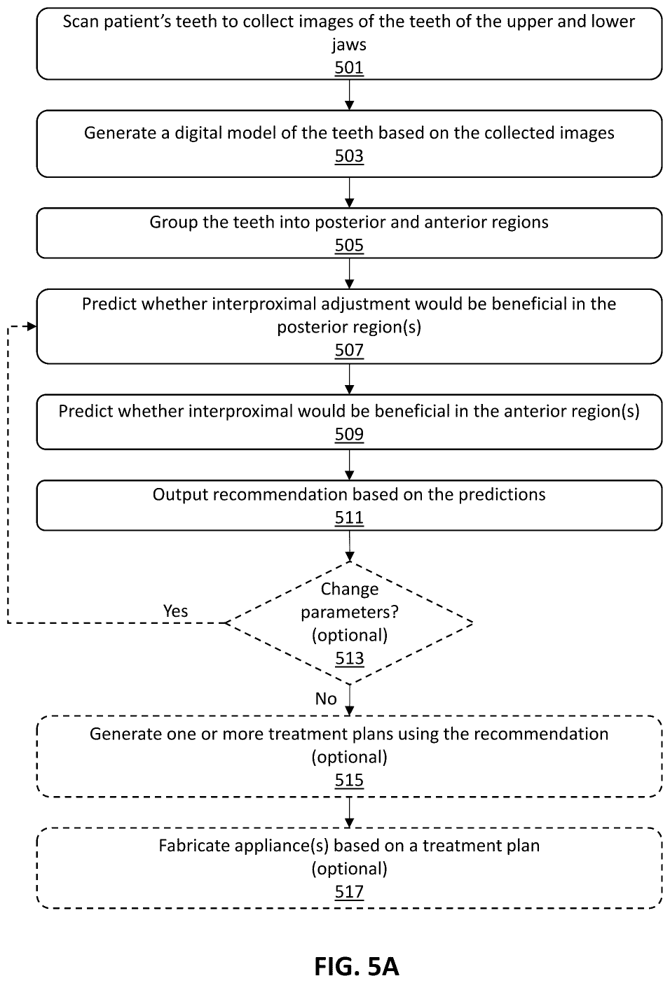

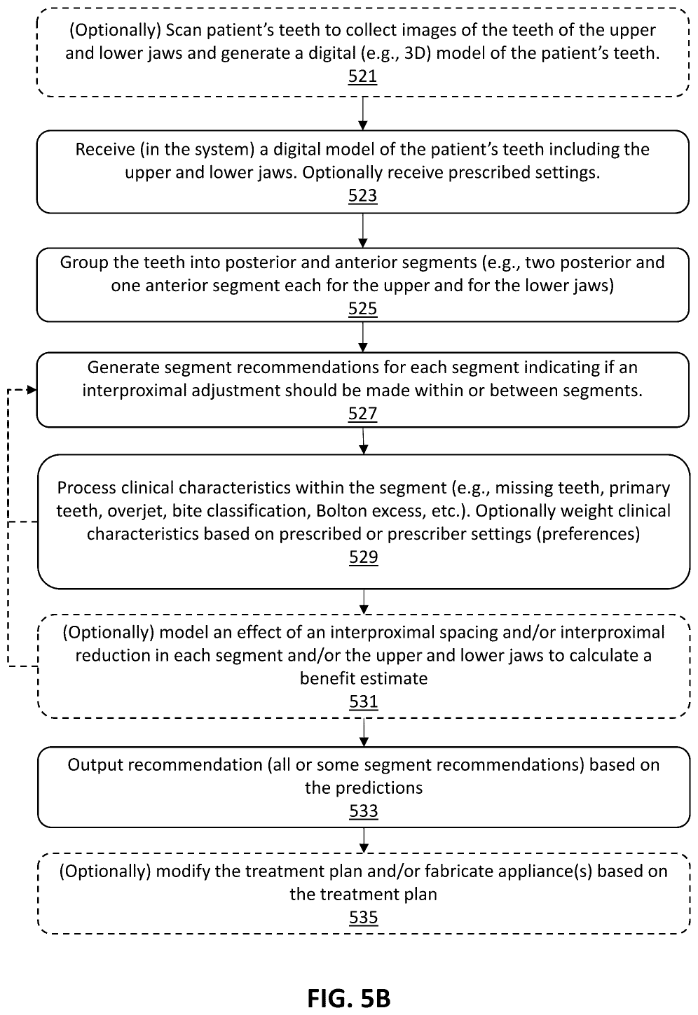

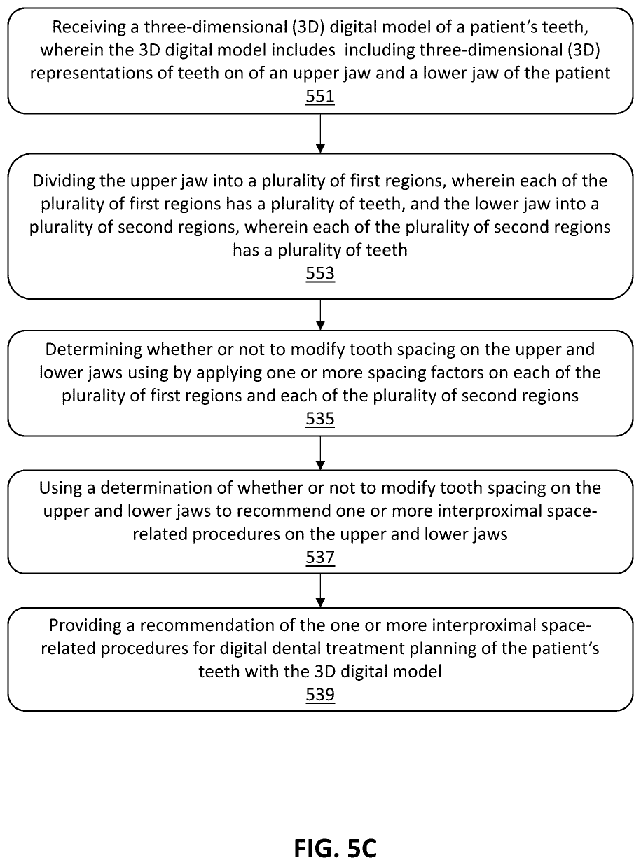

FIG. 5A illustrates one example of a method including predicting whether one or more interproximal adjustment (e.g., IPR and/or interproximal spacing) procedures would be beneficial as part of one or more orthodontic treatment plans. At 501, a patient's teeth are scanned to collect images of the teeth of the upper and lower jaws. In some cases, the images are collected using an intraoral scanner, which may be configured to direct radiation (e.g., visible light and/or ultraviolet (UV) radiation) toward the teeth and gingiva of the upper and lower jaws, and include one or more detectors configured to capture images of the teeth and gingiva. In some cases, the scan is performed immediately prior to analysis according to the flowchart of FIG. 5. In other cases, the scan is performed sometime prior to the analysis (e.g., day, weeks, months or years) and stored in memory for later retrieval and analysis.

At 503, a digital model of teeth of the upper and lower jaws is generated based on the captured images. The digital model may be a three-dimensional (3D) model generated from two-dimensional (2D) images collected by the intraoral scanner. In some examples, an image segmentation process is used to establish boundaries of each of the teeth and/or the gingiva in the digital model.

At 505, the teeth of the upper and lower jaws of the digital model are divided (e.g., by grouping teeth) into posterior and anterior segments. For example, the posterior segments may include including an upper left posterior segment, a lower left posterior segment, an upper right posterior segment and a lower right posterior segment, and the anterior segments may include an upper anterior segment and a lower anterior segment. Each of the posterior segments may include molars and premolars, and each of the anterior segments may include incisors and canine teeth.

At 507, a prediction/estimation as to whether one or more interproximal adjustments (e.g., IPR and/or interproximal spacing) in one or more of the posterior segments would be beneficial as part of a treatment plan is made. Sec, e.g., FIG. 2. This analysis may depend on whether a user (e.g., dental professional) desires an improvement in the molar class. If so, the prediction may involve determining any width discrepancies between the upper and lower left posterior segments, between the upper and lower right posterior segments, and between the upper and lower jaws. For example, the prediction as to whether one or more interproximal adjustments in one or more of the posterior segments would be beneficial may be based on an extent of any width discrepancies between matching upper and lower posterior segments and/or width discrepancies between the upper and lower jaws. If the extent of any width discrepancies is great enough (e.g., at or above a predetermined tolerance(s)), the prediction result may be to perform one or more interproximal adjustments on that particular matching posterior segment. If the user (e.g., dental professional) does not desire an improvement in the molar class, the prediction as to whether one or more interproximal adjustments in one or more of the posterior segments would be beneficial may be based on an extent of any initial (prior to implementation of the orthodontic treatment plan) bite class malocclusions of opposing canine teeth (e.g., class I, class II or class II initial canine bite class).

The analysis of 507 may be performed based on one or more inputs from a user (e.g., dental professional). For example, the user may be asked to provide input as to whether IPR on posterior regions is allowed (e.g., 200, FIG. 2), and/or whether it is desirable to improve a molar bite class (e.g., 204, FIG. 2), among other possible user inputs. In some cases, a graphical user interface (GUI) is presented to the user so that the user may easily enter the input. For example, the GUI may present one or more specific questions to the user that the user may respond to. In some examples, all or a portion of the decision tree for predicting whether one or more interproximal adjustments in one or more of the posterior regions would be beneficial (e.g., FIG. 2) is presented in the GUI. In some cases, one or more images are presented to the user. For example, 2D and/or 3D images of teeth (e.g., generic teeth and/or the particular patient's teeth) graphically indicating the questions as they related to the teeth may be presented to assist the user.

At 509, a prediction/estimation as to whether one or more interproximal adjustments (e.g., IPR and/or interproximal spacing) in one or more of the anterior segments (upper or lower) would be beneficial as part of a treatment plan is made. Sec, e.g., FIGS. 3A-3H. This analysis may be based on whether there are any missing or extracted canine teeth in the initial dentition (e.g., prior to treatment) and/or a predicted bite class of opposing canine teeth after the implementation of the orthodontic treatment plan. The predicted canine class may be determined based on factors related to the initial dentition and factors related to the expected treatment of the teeth. Sec, e.g., FIGS. 3B and 3C. Examples factors may include whether the patient has a mixed dentition (combination of primary and permanent teeth), whether there are any missing canine teeth in the initial dentition, whether any canine teeth are expected to be extracted during treatment, whether an anterior-proximal relationship is to be improved during treatment, whether any bicuspid teeth are expected to be extracted during treatment, the initial bite class of the canine teeth, whether any lower incisors are expected to be extracted, and/or a malocclusion (if any) severity of the initial bite class of the canine teeth. Further analysis may be made based on the predicted canine bite class. Sec, e.g., FIGS. 3D-3H. For example, prediction results may depend on whether there is a Bolton excess (when anterior maxillary teeth have relatively large mesio-distal widths compared to the mesio-distal widths of the anterior mandibular teeth) and the severity of the Bolton excess (e.g., at or below one or more predetermined values). In some examples, factors such as whether an overjet is to be maintained and/or whether the initial dentition has a negative overjet may be considered.

The analysis of 509 may be performed based on one or more inputs from a user (e.g., dental professional). For example, the user may be asked to provide input as to whether interproximal adjustments on anterior segments is allowed (e.g., 300, FIG. 3A), whether any canine teeth are expected to be extracted during treatment (e.g., 307, FIG. 3B), whether it is desirable for anterior-posterior relationship improvement and/or whether a bicuspid is to be extracted (e.g., 309, FIG. 3B), whether posterior IPR is allowed (e.g., 311, FIG. 3B), whether a lower incisor extraction is expected (e.g., 315, FIG. 3C and 324, FIG. 3F), and/or whether an overjet is to be maintained and/or if the overjet is negative (e.g., 314, FIG. 3E), among other possible user inputs. In some cases, these questions are gathered via the GUI as described above. The GUI may present one or more specific questions to the user that the user may respond to, and/or present all or a portion of the decision tree for predicting whether one or more interproximal adjustments in one or more of the anterior segments would be beneficial (e.g., FIGS. 3A-3H). In some cases, one or more images are presented to the user. For example, 2D and/or 3D images of teeth (e.g., generic teeth and/or the particular patient's teeth) graphically indicating the questions as they related to the teeth may be presented to assist the user.

At 511, an output with a recommendation to perform one or more interproximal adjustments in one or more of the posterior segments (e.g., lower left posterior segment, upper left posterior segment and/or lower right posterior segment) and/or one or more of the anterior segments (e.g., lower anterior segment and/or upper anterior segment) based on the predictions made at 507 and 509 is provided. The recommendation may be presented via the GUI, printed (e.g., on paper) and/or saved (and/or sent) as a digital file. In some cases, the digital file is in a format that is compatible with a related or separate treatment planning software program. The recommendation may be presented as a list or table indicating the type of recommended adjustment(s) (IPR and/or interproximal spacing) and/or where (e.g., which posterior and/or anterior segment) the interproximal adjustment(s) is/are recommended. In some examples, the recommendation may be associated with one or more images presented to the user. For example, images (e.g., 2D and/or 3D images) of teeth (e.g., generic teeth and/or the particular patient's teeth) indicating the type of adjustment(s) (IPR and/or interproximal spacing) and/or where (e.g., which posterior and/or anterior segment) the interproximal adjustment(s) is/are recommended. In some examples, all or part of analysis decision trees (e.g., FIGS. 2 and 3A-3H) are presented to the user.

At 513, the user may optionally choose to change one or more input parameters in the analyses of 507 and 509. For example, the user may decide to change one or more of the inputs provided in the prediction analysis of whether to perform IPR on one or more posterior segments of the teeth (FIG. 2) and/or the prediction analysis of whether to perform IPR and/or interproximal spacing on one or more anterior segments of the teeth (FIGS. 3A-3H). If the user decides to change one or more of the inputs (“Yes”), the analyses 507 and 509 may be repeated, resulting in a new output recommendation 511 based on the new user input. If the user decides not to change one or more of the inputs (“No”), the process may optionally proceed to step 515.

At 515, one or more treatment plans are optionally generated using the recommendation at 511. The treatment plan(s) may be based on incrementally moving the teeth from an initial teeth arrangement toward a target teeth arrangement by a series of stages. Each of the stages of a treatment plan may be associated with a removable orthodontic appliance (e.g., aligner and/or palatal expander) that is shaped and sized to reposition the teeth in accordance with a corresponding stage of the treatment plan. Thus, a patient may wear a series of removable orthodontic appliances to incrementally urge the teeth toward the target teeth arrangement. As described herein, an interproximal adjustment procedure may involve a procedure separate from the teeth repositioning implemented by removable orthodontic appliances (e.g., aligners and/or removable palatal expanders), which may be performed before, during and/or after treatment using the removable orthodontic appliances. An interproximal adjustment may include an IPR procedure (e.g., to reduce the size of one or more adjacent teeth) and/or an interproximal spacing procedure (e.g., to provide space between adjacent teeth for tooth-like material). For example, IPR may involve removing the enamel between adjacent teeth (e.g., to correct crowding and/or or reshape the contact area between the adjacent teeth), and interproximal spacing may be follow up with bonding tooth-like material to one or more of the adjacent teeth.

If the recommendation is to perform an IPR procedure, a treatment plan may include a planned movement of adjacent teeth applied by one or more removable orthodontic appliances to anticipate the IPR. This may involve allowing adjacent teeth to move closer together using the dental appliance(s) than would normally be allowed if an IPR was not part of a treatment plan. If the recommendation is to perform an interproximal spacing procedure, a treatment plan may include a planned movement of adjacent teeth applied by one or more removable orthodontic appliances to anticipate the interproximal spacing procedure. This may involve spreading adjacent teeth farther apart from each other (e.g., to provide space for tooth-like material) using the dental appliance(s) than would normally be allowed if an interproximal spacing procedure was not part of a treatment plan. Thus, the recommendation may be integrated into the treatment plan to prescribe appropriate repositioning of the teeth.

In some cases, multiple treatment plans may be generated from the recommendation. For example, there may be more than one way to reposition the teeth to anticipate IPR and/or interproximal spacing in one or more of the posterior and/or anterior segments. In such cases, the user may be provided the option to choose a particular treatment plan from the multiple treatment plans. Aspects of each of the multiple treatment plans, such as length of treatment and treatment difficulty, may be presented to the user (e.g., via a GUI) to assist the user in choosing a particular treatment plan.

At 517, one or more of the orthodontic appliances may be fabricated based on a chosen treatment plan. For example, a series of orthodontic appliances may be fabricated based on the incremental treatment plan. In some cases, multiple series of orthodontic appliances are fabricated based on multiple treatment plans. In some examples, the orthodontic appliances are at least partially made of a polymeric material having cavities that are configured to receive teeth of the patient's dentition. In some examples, the appliances may be manufactured using a direct fabrication technique, such as additive manufacturing (e.g., 3D printing). Direct fabrication may involve forming the appliances based on 3D digital models of the appliances. Alternatively or additionally, the appliances may be manufactured using a molding process. For example, a 3D mold of the dentition at the various treatment stages may be formed (e.g., based on 3D digital models of the dentition), and a polymer material may be molded on the 3D molds of the dentition to form the appliances.

FIG. 5B illustrates an example of a method as described herein, which may also include generating a segment recommendation for each segment of the plurality of segments indicating if one or more of interproximal adjustment procedures should be performed as part of an orthodontic treatment plan.

For example, In FIG. 5B, the method may optionally include generating the digital model of the patient's upper and lower jaws, for example by scanning, e.g., using an intraoral scanner, the patient's upper and lower jaws 521. Alternatively, the digital model of the patient's upper and lower jaw may be received from another source. The digital model may be pre-processed, including by segmentation to identify and/or separate individual teeth from the digital model.

In any of these method the digital model may be received 523, e.g., by the system performing the method, which may include one or more processors. In some examples, received may include receiving from an external source (such as from an intraoral scanner, or a separate and/or networked system or sub-system that generates the digital model). In some examples received may include receiving from a sub-system within the same system (e.g., from a separate module or engine).

The digital model of the upper and lower jaw may then be divided into segments, e.g., by grouping teeth from the upper jaw into three (or more) segment and grouping teeth from the lower jaw into three (or more) segments 525. For example, grouping teeth of the digital model into a plurality of segment may include grouping into at least two posterior segments and at least one anterior segment for each of the upper jaw and the lower jaw, wherein each of the posterior segments includes at least one molar and at least one premolar, and each of the anterior segments includes at least one incisor and at least one canine tooth.

The method may then generate a segment recommendation for each segment (or in some examples between segments) of the plurality of segments indicating if one or more of interproximal adjustment procedures should be performed as part of an orthodontic treatment plan 527, as described herein. For example, the method or apparatus may include processing clinical characteristics within each segment (e.g., presence of missing teeth, a presence of primary teeth, a degree of overjet, a bite classification of the teeth, and a degree of Bolton excess of the teeth, etc.) 529. Optionally weight clinical characteristics based on prescribed or prescriber settings (preferences). Thus, the value of the clinical characteristic may be weighted by the prescribed settings specific to a particular patient and/or prescriber setting, e.g., generally applied by the user (dental professional) associated with that patient. Prescribed settings may overrule prescriber preferences. In any of these examples generating the recommendation may include modeling an effect of an interproximal spacing and/or interproximal reduction in each segment and/or the upper and lower jaws to calculate a benefit estimate 531, as described herein. The steps of generating the segment recommendations may be performed in parallel and/or in series; if preformed in series, they may be repeated for all of the segments.

Any of these methods may include outputting the segment recommendations (or a consolidated recommendation including all or some of the segment recommendations) 533. In any of these example the method may include modifying the treatment plan and/or fabricating one or more (e.g., a series of) appliances based on the modified treatment plan 535.

FIG. 5C illustrates another example of a method as described herein. In FIG. 5C, the method includes: receiving a three-dimensional (3D) digital model of a patient's teeth, wherein the 3D digital model includes including three-dimensional (3D) representations of teeth on of an upper jaw and a lower jaw of the patient 551. As described above, the method may further include dividing the upper jaw into a plurality of first regions, wherein each of the plurality of first regions has a plurality of teeth, and the lower jaw into a plurality of second regions, wherein each of the plurality of second regions has a plurality of teeth 553.

As shown in FIG. 3C, the method may further include determining whether or not to modify tooth spacing on the upper and lower jaws using by applying one or more spacing factors on each of the plurality of first regions and each of the plurality of second regions 535 and using a determination of whether or not to modify tooth spacing on the upper and lower jaws to recommend one or more interproximal space-related procedures on the upper and lower jaws 537. Any of these methods may further include providing a recommendation of the one or more interproximal space-related procedures for digital dental treatment planning of the patient's teeth with the 3D digital model 539.

In any of the method described herein the recommendation may be the individual (or collected/grouped) segment recommendations, or an output based on or incorporating all or some of the segment recommendations, including a digital model, e.g., of the patient's teeth, showing the recommended interproximal adjustment and/or showing (e.g., modeling) the predicted outcome of the recommended interproximal adjustment.

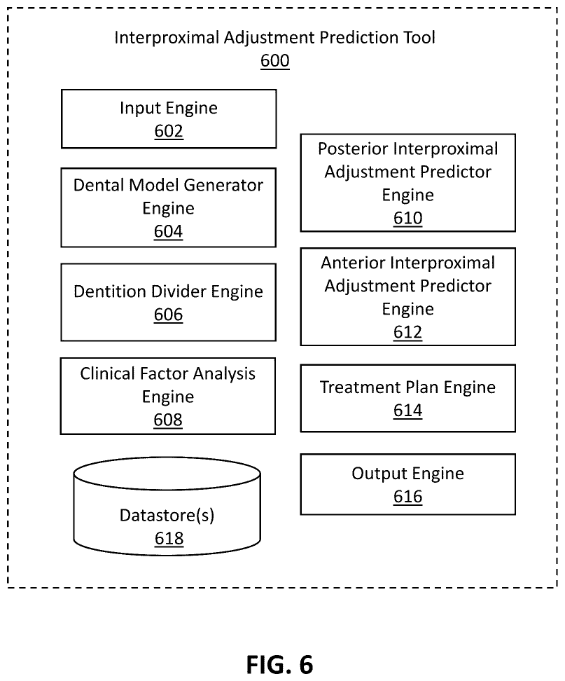

FIG. 6 is a diagram showing an example interproximal adjustment prediction tool 600. The interproximal adjustment prediction tool 600 may be incorporated into a portion of one or more treatment planning systems and/or one or more dental appliance manufacturing systems, and may therefore also be referred to as a sub-system. In other cases, the interproximal adjustment prediction tool 600 may be a stand-alone tool or an add-on (e.g., plug-in) to one or more treatment planning systems and/or one or more dental appliance manufacturing systems. In any of the methods and apparatuses described herein, the interproximal adjustment prediction tool 600 may be invoked by a user control, such as a tab, button, etc., as part of a treatment planning system and/or dental appliance manufacturing system, or may be separately invoked.

The interproximal adjustment prediction tool 600 includes engines. As used herein, an engine includes one or more processors or a portion thereof. A portion of one or more processors may include some portion of hardware less than all of the hardware including any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine may have one or more dedicated processors, or a first engine and a second engine can share one or more processors with one another or other engines. The engines may all use the same processor(s) (e.g., as part of one computer) or may use different processors (e.g., as part of multiple computers). Depending upon implementation-specific or other considerations, an engine may be centralized, or its functionality distributed. An engine may include hardware, firmware, or software embodied in a computer-readable medium for execution by the processor. The processor transforms data into new data using implemented data structures and methods, such as is described with reference to the figures herein.

One or more datastores 618 may include data structures for storing data. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastore(s) 618 may be cloud-based datastores. A cloud-based datastore is a datastore that is compatible with cloud-based computing systems and engines.

The interproximal adjustment prediction tool 600 may include or be part of a computer-readable medium and may include an input engine 602 that allows access to data. For example, the input engine 602 may provide access to data related to a patient and/or a library of data. Such data may include a patient's dental scan data (e.g., image data). The scan data may include 2D image data related to the capture of images of the patient's oral cavity (e.g., teeth, gingiva, bone and/or soft tissue). All or some of the data may be stored in the datastore(s) 618.

A dental model generator engine 604 is configured to generate one or more digital models of one or more dentitions. The models may include 2D and/or 3D digital models of portions of a patient's oral cavity based on received image data. In some cases, the dental model generator engine 604 is configured to perform an image segmentation process in which different segments of the model(s) are partitioned into image segments to establish boundaries between teeth and gingiva (and possibly other tissue types) in the digital model. In some cases, the image segmentation process defines boundaries of each tooth of a patient's dentition. In some instances, digital model generator engine 604 is configured to identify and label each tooth of the patient's dentition.

A dentition divider engine 606 is configured to group teeth of the digital model(s) of dentition(s) into segments, where each segment includes multiple teeth. For example, a dentition may be divided into posterior segments (e.g., upper left posterior segment, lower left posterior segment, upper right posterior segment and lower right posterior segment) and anterior segments (e.g., upper anterior segment and lower anterior segment). In some examples, each of the posterior segments includes molar and premolars, and each of the anterior segments includes incisors and canine teeth. In some instances, the dividing process includes identifying the type of each tooth (molar, premolar, incisor, canine), identifying the jaw in which each tooth resides (lower or upper), identifying the side of the jaws in which each tooth resides (left or right), and grouping the teeth together based on tooth type, jaw and side.

A clinical factor analysis engine 608 is configured to analyze and characterize one or more clinical factors of the digital dental model. Such clinical factors may include one or more of: overjet identification including type and extent (e.g., FIG. 1A), bite class identification (e.g., of canines, molars and/or premolars) (e.g., FIG. 1B), Bolton analysis (e.g., FIG. 1C) including identification and an extent of Bolton excess, identification of any missing teeth, and identification of any primary teeth (for mixed dentition). In some examples, the analyses and characterization are performed based on computer analysis of the digital dental model alone (e.g., without input from a user). In other examples, the analyses and characterization are performed based on computer analysis of the digital dental model and input from a user.

A posterior interproximal adjustment predictor engine 610 is configured to predict/estimate whether one or more interproximal adjustment procedures (e.g., IPR and/or interproximal spacing) for each of the posterior segments identified by the dentition divider engine 606 would be beneficial as part of an orthodontic treatment plan. The posterior interproximal adjustment predictor engine 608 may be configured to consider one or more of the clinical factors of the digital dental model characterized by the clinical factor analysis engine 608 and other factors related to treatment plan expectations (e.g., desired outcome, treatment time, etc.) to make a prediction as to whether one or more interproximal adjustment procedures would be beneficial. The posterior interproximal adjustment predictor engine 610 may use any of a number of analyses techniques, such as regression analysis(es), decision tree analysis(es) and/or machine learning (e.g., ensemble learning, support vector machine, etc.). In some instances, the posterior interproximal adjustment predictor engine 610 implements a decision tree analysis such as shown in FIG. 2 and described above.

An anterior interproximal adjustment predictor engine 612 is configured to predict/estimate whether one or interproximal adjustment procedures (e.g., IPR and/or interproximal spacing) for each of the anterior segments identified by the dentition divider engine 606 would be beneficial as part of an orthodontic treatment plan. The anterior interproximal adjustment predictor engine 612 may be configured to consider one or more of the clinical factors of the digital dental model characterized by the clinical factor analysis engine 608 and other factors related to treatment plan expectations (e.g., desired outcome, treatment time, etc.) to make a prediction as to whether one or more interproximal adjustment procedures would be beneficial. The anterior interproximal adjustment predictor engine 612 may use any of a number of analyses techniques, such as regression analysis(es), decision tree analysis(es) and/or machine learning (e.g., ensemble learning, support vector machine, etc.). In some instances, the anterior interproximal adjustment predictor engine 612 implements a decision tree analysis such as shown in FIGS. 3A-3H and described above.

A treatment plan engine 614 is configured to generate one or more orthodontic treatment plans to treat the patient's dentition. As described herein, the orthodontic treatment plans may be divided into multiple stages designed to incrementally urge the teeth toward a target tooth arrangement using multiple dental appliances (e.g., aligners) corresponding to the multiple stages. This may involve calculating repositioning forces on the teeth and/or expansion forces on the dental arch(es). These calculations may include determining the direction and magnitude of forces applied to each of the teeth to achieve the target tooth arrangement. The treatment plan engine 614 may directly or indirectly (e.g., via user input) use one or more recommendation results from the posterior interproximal adjustment predictor engine 610 and/or anterior interproximal adjustment predictor engine 612 to incorporate one or more posterior and/or anterior interproximal adjustments planned to be implemented before, during or after treatment using the dental appliances.

An output engine 616 is configured to generate one or more data outputs. The output engine 616 may access data stored in the datastore 618, such as 3D model data related to the dentition or the dental appliances for each stage of the treatment plan. The format of the data output may depend on the type of output. For example, image format data may be used to display image data on one or more computer screens. In some cases, data is in a format that is readable by an additive manufacturing machine (e.g., 3D printer) to fabricate physical dental appliances (e.g., polymer aligners). In some cases, the output may include instructions fabricating the dental appliances. In some cases, the output may be in a format suitable for forming the dental appliances, or a portion thereof, using one or more manufacturing processes other than additive manufacturing, such as a molding process or etching process.

The engines described herein may include cloud-based engines. As used herein, a cloud-based engine is an engine that can run applications and/or functionalities using a cloud-based computing system. All or portions of the applications and/or functionalities may be distributed across multiple computing devices and need not be restricted to only one computing device. In some embodiments, the cloud-based engines may execute functionalities and/or modules that end users access through a web browser or container application without having the functionalities and/or modules installed locally on the end-users' computing devices.

The interproximal adjustment prediction tool 600 may include a computer-readable medium. The modules/engines may be coupled to one another in any of a number of ways. The modules/engines may be coupled to modules/engines not explicitly shown in FIG. 6. The computer-readable medium may include any computer-readable medium, including without limitation, a bus, a wired network, a wireless network, or some combination thereof. The engines described herein may implement one or more automated agents, including machine learning agents.

The datastores 618 include repositories having any applicable organization of data, including tables, comma-separated values (CSV) files, traditional databases (e.g., SQL), or other applicable known or convenient organizational formats. Datastore(s) 618 may be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

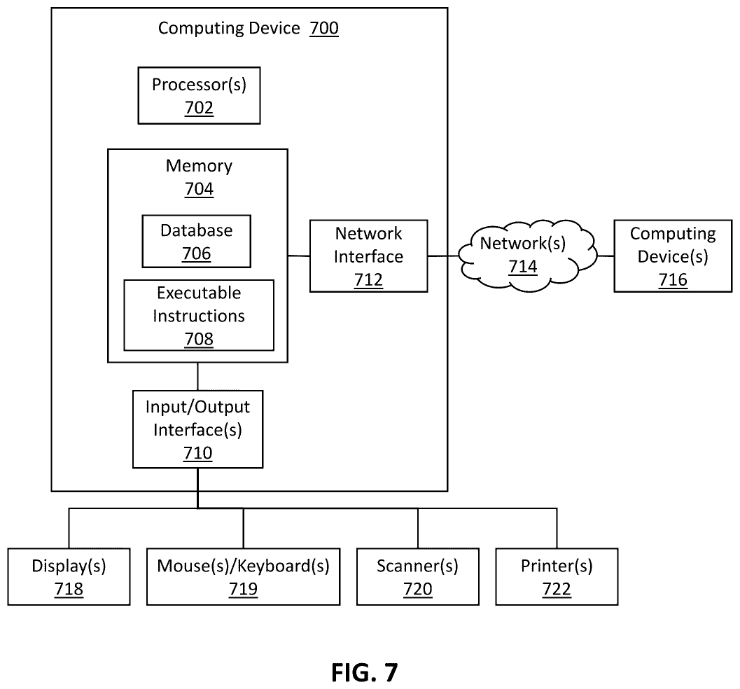

FIG. 7 shows an example computing device 700 configured to perform methods described herein. The computing device 700 device may be part of a scanning system for scanning a patient's oral cavity (e.g., intraoral scanning system). Alternatively, the computing device 700 may be separate from a scanning device/system. The computing device 700 includes one or more processors 702 and memory 704. The memory 704 may include various types of information including data 706 and computing device executable instructions 708 as discussed herein. For example, the memory 704 may be used to store executable instructions 708 that may be used to interact with the other components of the computing device 700 and/or one or more other computing devices 716 via one or more networks 714 and may be used to store information, such as instructions for manipulating one or more files.

The computing device 700 may include executable instructions 708 for saving a number of program and/or data files, such as files, for providing executable instructions 708 that allow for the viewing functionality for viewing scans and/or models, and the data files for the scans and/or digital models. Some executable instructions 708 may, for example, be instructions for saving local scans and/or digital models, scans and/or digital models from another computing device 716 on the network 714, or a combination of two or more of these.

As illustrated in FIG. 7, the computing device 700 may include a network interface 712. The interface 712 may allow for processing on one or more networked computing devices 716, or the computing devices 716 may be used to transmit and/or receive scans and/or digital models and/or executable instructions for use with various methods described herein. In some examples, the networked computing devices 716 may include those that are associated with one or more manufacturing systems. As described herein, the manufacturing systems(s) may include additive manufacturing equipment (e.g., 3D printer(s)), molding equipment, and/or the like.

As illustrated, the computing device 700 may include one or more input and/or output interfaces 710. Such interfaces 710 may be used to connect the computing device 700 with one or more input and/or output devices. Examples input and output devices may include one or more displays 718 (e.g., computer monitor or screen), one or more mouses and/or keyboards 719, one or more scanners 720 and/or one or more printers 722.

The one or more scanners 720 may include an intraoral scanner that is configured to collect images of the inside of a patient's mouth. The scanner(s) 720 may include one or more radiation (e.g., visible, infrared, near infrared and/or x rays) sources and one or more sensors/detectors for detecting radiation that is reflected and/or scattered off tissue of the patient's mouth. In some cases, the scanner(s) may include a wand that is shaped and sized to fit at least partially within the patient's mouth. The wand may be configured to direct the radiation into the patient's mouth and collect reflected and/or scattered radiation to collect images of the patient's mouth. In some examples, components of the scanner(s) are controlled by the computing device 700. For example, the executable instructions 708 may be configured to cause the processor(s) 702 to control aspects of image collection of the scanner(s), and the image data may be stored in the memory 704 as data 706. Alternatively, the scanner(s) may be controlled by a computing device separate from the computing device 700. Data from a scan of the patient's teeth may be used to form a digital model (e.g., 3D digital model) of the patient's teeth, which may be used to identify and characterize malocclusions and to use as a basis for generating one or more treatment plans, as described herein.

The one or more printers 722 may include one or more 3D printers configured to fabricate dental appliance(s), such as polymeric aligners, based on one or more treatment plans. Alternatively or additionally, the one or more printers 722 may include one or more ink-based printers that is/are configured to print images and/or text on paper or other material.

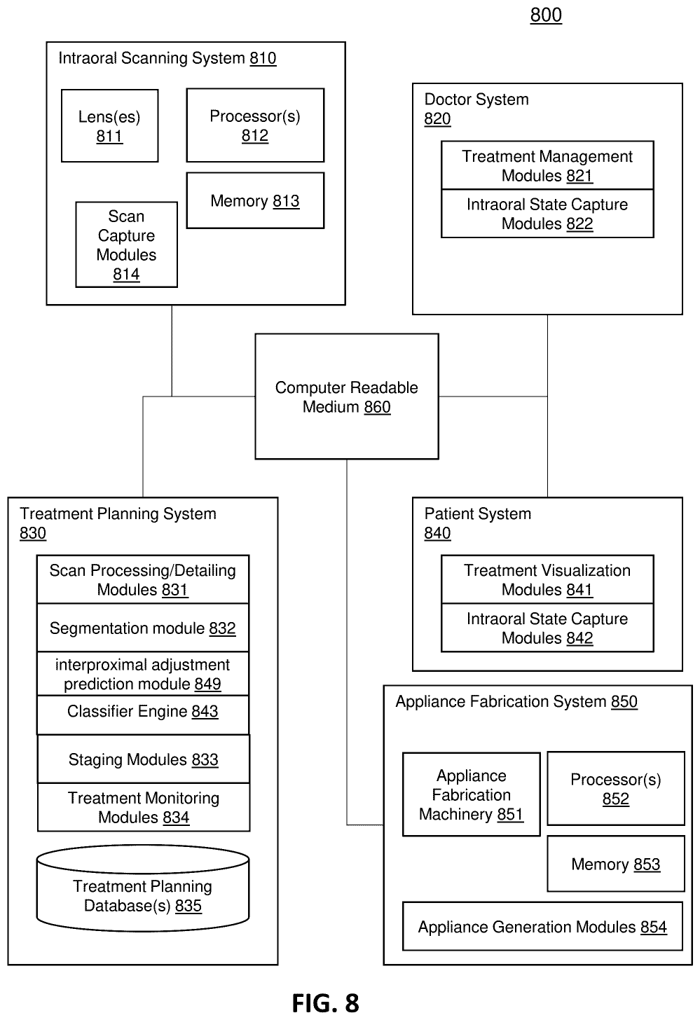

FIG. 8 is a diagram illustrating one variation of a computing environment 800 that may generate one or more orthodontic/dental appliances and/or treatment plans specific to a patient, and fabricate dental appliances that may accomplish the treatment plan to treat a patient, under the direction of a dental professional. In general, one or more component of the computing module may include an interproximal adjustment prediction module configured to perform any of the methods described herein. The example computing environment 800 shown in FIG. 8 includes an intraoral scanning system 810, a doctor system 820, a treatment planning system 830 (e.g., technician system), a patient system 840, an appliance fabrication system 850, and computer-readable medium 860. Each of these systems may be referred to equivalently as a sub-system of the overall system (e.g., computing environment). Although shown as discrete systems, some or all of these systems may be integrated and/or combined. In some variations a computing environment (dental computing system) 800 may include just one or a subset of these systems (which may also be referred to as sub-systems of the overall system 800). As mentioned, one or more of these systems may be combined or integrated with one or more of the other systems (sub-systems), such as, e.g., the patient system and the doctor system may be part of a remote server accessible by doctor and/or patient interfaces. The computer readable medium 860 may divided between all or some of the systems (subsystems); for example, the treatment planning system and appliance fabrication system may be part of the same sub-system and may be on a computer readable medium 860. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, an intraoral scanning system 810 can include optics 811 (e.g., one or more lenses, filters, mirrors, etc.), processor(s) 812, a memory 813, and a scan capture module 814. In general, the intraoral scanning system 810 can capture one or more images of a patient's dentition. Use of the intraoral scanning system 810 may be in a clinical setting (doctor's office or the like) or in a patient-selected setting (the patient's home, for example). In some cases, operations of the intraoral scanning system 810 may be performed by an intraoral scanner, dental camera, cell phone or any other feasible device.

The optical components 811 may include one or more lenses and optical sensors to capture reflected light, particularly from a patient's dentition. The scan capture module 814 can include instructions (such as non-transitory computer-readable instructions) that may be stored in the memory 813 and executed by the processor(s) 812 to can control the capture of any number of images of the patient's dentition.

In FIG. 8 the segmentation 832 and verification 832 (e.g., classifier engine 843) are shown as part of the treatment planning sub-system 830, however in some examples some, or all, of these components may be part of (or duplicated in) the intraoral scanning system 810. For example, the segmentation module 832 may be in the intraoral scanning sub-system 810 or another sub-system. Any of the component systems or sub-systems of the dental computing environment 800 may access or use the segmented data. Similarly, in FIG. 8, the interproximal adjustment prediction module(s) 489 are shown as part of the treatment planning sub-system 830; the interproximal adjustment prediction module(s) may be part of, accessed by, or duplicated in, one or more other sub-systems, including the doctor system 820, scanning system 810, fabrication system 850, etc.

The doctor system 820 (e.g., doctor sub-system) may include a treatment management module 821 and an intraoral state capture module 822 that may access or use the 3D model based on the segmented data. The doctor system 820 may provide a “doctor facing” interface to the computing environment 800. The treatment management module 821 can perform any operations that enable a doctor or other clinician to manage the treatment of any patient. In some examples, the treatment management module 821 may provide a visualization and/or simulation of the patient's dentition with respect to a treatment plan. This user interface may also include display the segmentation.

The intraoral state capture module 822 can provide images of the patient's dentition to a clinician through the doctor system 820. The images may be captured through the intraoral scanning system 810 and may also include images of a simulation of tooth movement based on a treatment plan.

In some examples, the treatment management module 821 can enable the doctor to modify or revise a treatment plan, particularly when images provided by the intraoral state capture module 822 indicate that the movement of the patient's teeth may not be according to the treatment plan. The doctor system 820 may include one or more processors configured to execute any feasible non-transitory computer-readable instructions to perform any feasible operations described herein.

Alternatively or additionally, the treatment planning system 830 may include any of the methods and apparatuses described herein. The treatment planning system 830 may include scan processing/detailing module 831, segmentation module 832, classifier engine(s) 843, staging module 833, and treatment monitoring module 834, interproximal adjustment prediction module 849, and a treatment planning database(s) 835. In general, the treatment planning system 830 can determine a treatment plan for any feasible patient. The scan processing/detailing module 831 can receive or obtain dental scans (such as scans from the intraoral scanning system 810) and can process the scans to “clean” them by removing scan errors and, in some cases, enhancing details of the scanned image. The treatment planning system 830 may perform segmentation. For example, a treatment planning system may include a segmentation module 832 that can segment a dental model into separate parts including separate teeth, gums, jaw bones, and the like. In some cases, the dental models may be based on scan data from the scan processing/detailing module 831 and/or segmentation data from the segmentation module (and/or classifier engine 843).

The interproximal adjustment prediction module(s) 849 may be configured to operate as the device 700 shown in FIG. 7, discussed above, and/or as the prediction tool 600, as described in reference to FIG. 6.

The staging module 833 may determine different stages of a treatment plan. Each stage may correspond to a different dental aligner. The staging module 833 may also determine the final position of the patient's teeth, in accordance with a treatment plan. Thus, the staging module 833 can determine some or all of a patient's orthodontic treatment plan. In some examples, the staging module 833 can simulate movement of a patient's teeth in accordance with the different stages of the patient's treatment plan.

An optional treatment monitoring module 834 can monitor the progress of an orthodontic treatment plan. In some examples, the treatment monitoring module 834 can provide an analysis of progress of treatment plans to a clinician. Although not shown here, the treatment planning system 830 can include one or more processors configured to execute any feasible non-transitory computer-readable instructions to perform any feasible operations described herein.

The patient system 840 can include a treatment visualization module 841 and an intraoral state capture module 842. In general, the patient system 840 can provide a “patient facing” interface to the computing environment 800. The treatment visualization module 841 can enable the patient to visualize how an orthodontic treatment plan has progressed and also visualize a predicted outcome (e.g., a final position of teeth).

In some examples, the patient system 840 can capture dentition scans for the treatment visualization module 841 through the intraoral state capture module 842. The intraoral state capture module can enable a patient to capture his or her own dentition through the intraoral scanning system 810. Although not shown here, the patient system 840 can include one or more processors configured to execute any feasible non-transitory computer-readable instructions to perform any feasible operations described herein.

The appliance fabrication system 850 can include appliance fabrication machinery 851, processor(s) 852, memory 853, and appliance generation module 854. In general, the appliance fabrication system 850 can directly or indirectly fabricate aligners to implement an orthodontic treatment plan. In some examples, the orthodontic treatment plan may be stored in the treatment planning database(s) 835. Any of these apparatuses and methods may be configured to include the step of fabricating one or more (e.g., a series) of dental appliances using a 3D model (e.g., including using the corrected segmentation, as described herein).

The appliance fabrication machinery 851 may include any feasible implement or apparatus that can fabricate any suitable dental aligner. The appliance generation module 854 may include any non-transitory computer-readable instructions that, when executed by the processor(s) 852, can direct the appliance fabrication machinery 851 to produce one or more dental aligners. The memory 853 may store data or instructions for use by the processor(s) 852. In some examples, the memory 853 may temporarily store a treatment plan, dental models, or intraoral scans.

The computer-readable medium 860 may include some or all of the elements described herein with respect to the computing environment 800. The computer-readable medium 860 may include non-transitory computer-readable instructions that, when executed by a processor, can provide the functionality of any device, machine, or module described herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-37. (canceled)

38. A method comprising:
receiving a three-dimensional (3D) digital model of a patient's teeth, wherein the 3D digital model includes including three-dimensional (3D) representations of teeth on an upper jaw and a lower jaw of the patient;
dividing the upper jaw into a plurality of first segments, wherein each of the plurality of first segments has a plurality of teeth, and the lower jaw into a plurality of second segments, wherein each of the plurality of second segments has a plurality of teeth;
determining whether or not to modify tooth spacing on the upper and lower jaws using by applying one or more spacing factors on each of the plurality of first segments and each of the plurality of second segments;
using a determination of whether or not to modify tooth spacing on the upper and lower jaws to recommend one or more interproximal space-related procedures on the upper and lower jaws; and
performing the one or more interproximal space-related procedures on the upper and lower jaws, wherein performing comprises providing a recommendation of the one or more interproximal space-related procedures as part of a digital dental treatment planning of the patient's teeth with the 3D digital model.

39. The method of claim 38, wherein the determination whether or not to modify tooth spacing on the upper and lower jaws comprises one or more indications whether interproximal spaces are allowed or not on the plurality of first segments or the plurality of second segments.

40. The method of claim 38, wherein the determination whether or not to modify tooth spacing on the upper and lower jaws comprises an evaluation of the plurality of first or second segments against one or more prediction trees, wherein the one or more prediction trees comprise a plurality of decision nodes representing clinical criteria for evaluating spacing determinations on the plurality of first or second segments.

41. The method of claim 38, wherein:
the determination whether or not to modify tooth spacing on the upper and lower jaws comprises an evaluation of the plurality of first or second segments against one or more prediction trees, wherein the one or more prediction trees comprise a plurality of decision nodes representing clinical criteria for evaluating spacing determinations on the plurality of first or second segments; and
the prediction trees comprise one or more of: a posterior interproximal reduction (IPR) prediction tree, a canine class prediction tree, a mixed canine class decision tree, and an anterior IPR spacing prediction tree.

42. The method of claim 38, wherein the one or more spacing factors comprise one or more clinical setup characteristics for the patient's teeth.

43. The method of claim 38, wherein:
the one or more spacing factors comprise one or more clinical setup characteristics for the patient's teeth;
the one or more clinical setup characteristics comprise one or more of: a presence of missing teeth, a presence of primary teeth, a degree of overjet, a bite classification of the teeth, and a degree of Bolton excess of the teeth.

44. The method of claim 38, wherein the one or more spacing factors comprise one or more prescribed settings for the patient's teeth.

45. The method of claim 38, wherein:
the one or more spacing factors comprise one or more prescribed settings for the patient's teeth; and
the one or more prescribed settings comprise one or more of: settings to manage a interproximal reduction (IPR) procedure on the plurality of first segments or the plurality of second segments, settings to manage spaces on the plurality of first segments or the plurality of second segments, a bite class goal for the patient's teeth, and an overjet goal for the patient's teeth.

46. The method of claim 38, wherein:
the plurality of first segments comprise one or more of: a first posterior segment with a plurality of upper posterior teeth, and a plurality of first anterior segments, each with a plurality of upper anterior teeth;
the plurality of second segments comprise one or more of: a second posterior segment with a plurality of lower posterior teeth, and a plurality of second anterior segments, each with a plurality of lower anterior teeth;
or some combination thereof.

47. The method of claim 38, further comprising incorporating the recommendation of the one or more interproximal space-related procedures into a final position determination in a digital dental treatment plan.

48. The method of claim 38, further comprising using the recommendation of the one or more interproximal space-related procedures to recommend a stage of a digital treatment plan to perform the one or more interproximal space-related procedures.

49. The method of claim 38, further comprising displaying the recommendation of the one or more interproximal space-related procedures on a computer-implemented display.

50. The method of claim 38, further comprising displaying the recommendation of the one or more interproximal space-related procedures on a representation of the 3D digital model of the patient's teeth.

51. The method of claim 38, further comprising:
displaying the recommendation of the one or more interproximal space-related procedures on a representation of the 3D digital model of the patient's teeth; and
processing one or more user interactions related to the one or more interproximal space-related procedures with the representation of the 3D digital model of the patient's teeth.

52. The method of claim 38, further comprising:
taking an intraoral scan of the patient's teeth;
converting the intraoral scan into the 3D dental model.

53. The method of claim 38, further comprising exporting a digital treatment plan comprising the recommendation of the one or more interproximal space-related procedures for design of aligners to implement a digital treatment plan.

54. A system comprising:
one or more processors;
memory coupled to the one or more processors storing computer-program instructions that, when executed by the one or more processors, cause the system to perform:
receiving a three-dimensional (3D) digital model of a patient's teeth, wherein the 3D digital model includes including three-dimensional (3D) representations of teeth on of an upper jaw and a lower jaw of the patient;
dividing the upper jaw into a plurality of first segments, wherein each of the plurality of first segments has a plurality of teeth, and the lower jaw into a plurality of second segments, wherein each of the plurality of second segments has a plurality of teeth;
determine whether or not to modify tooth spacing on the upper and lower jaws using by applying one or more spacing factors on each of the plurality of first segments and each of the plurality of second segments; and
using a determination of whether or not to modify tooth spacing on the upper and lower jaws to recommend one or more interproximal space-related procedures on the upper and lower jaws;
providing a recommendation of the one or more interproximal space-related procedures for digital dental treatment planning of the patient's teeth with the 3D digital model.

55. The system of claim 54, further comprising a display coupled to the one or more processors and operative to display the recommendation of the one or more interproximal space-related procedures.

56. The system of claim 54, further comprising a display coupled to the one or more processors and operative to display the recommendation of the one or more interproximal space-related procedures on a representation of the 3D digital model of the patient's teeth.

57. The system of claim 54, further comprising an intraoral scanner configured to take a scan of the patient's teeth.

58. The system of claim 54, further comprising an intraoral scanner configured to take a scan of the patient's teeth, wherein the computer-program instructions, when executed by the one or more processors, cause the system to convert the intraoral scan into the 3D dental model.
